(19) United States
(12) Patent Application Publication
(10) Pub. No.: US 2025/0195832 A1
(43) Pub. Date: Jun. 19, 2025

(54) MEDICAL DEVICES

(71) Applicant: Unomedical A/S, Lejre (DK)

(72) Inventors: Erik Edwards, Columbus, OH (US); Rachel Zimet Pytel, Las Vegas, NV (US); Lisa Jaunet van Kooten, Lejre (DK)

(21) Appl. No.: 19/072,940

(22) Filed: Mar. 6, 2025

Related U.S. Application Data
(63) Continuation of application No. PCT/EP2023/073836, filed on Aug. 30, 2023.
(60) Provisional application No. 63/404,305, filed on Sep. 7, 2022.

(30) Foreign Application Priority Data
Oct. 20, 2022 (GB) 2215514.7

(51) Int. Cl. A61M 25/00; A61L 29/08; A61M 5/14; C08L 67/04; C08L 79/02; C08L 81/08

(52) U.S. Cl. CPC A61M 25/0045; A61L 29/085; A61M 5/14; C08L 67/04; C08L 79/02; C08L 81/08; A61M 2005/1401

(57) ABSTRACT

The invention provides a medical device comprising a fluoropolymer surface comprising at least one species independently chosen from: a carboxybetaine, a sulfobetaine, and combinations and/or polymers thereof.

### TECHNICAL FIELD OF THE INVENTION

The present invention relates to medical devices comprising fluoropolymer surfaces containing carboxybetaine and/or sulfobetaine species. The present invention also relates to the use of carboxybetaine and/or sulfobetaine species as protein-repellents in/on a medical device.

### BACKGROUND TO THE INVENTION

Cannulas and catheters are indispensable in the medical field and are inserted into the body, often for the delivery or removal of fluid. The material and configuration of such medical devices vary enormously depending on their intended use. Typical uses of cannulas and catheters include cardiovascular, urological, gastrointestinal, neurovascular, and ophthalmic applications.

There has been recent interest in constructing such medical devices using fluoropolymer materials, in particular polytetrafluoroethylene (PTFE). Such fluoropolymers are advantageous for use in medical applications due to their favourable mechanical properties and excellent chemical stability under biological conditions.

However, when such insertable medical devices are introduced into the body, foreign body responses can occur—i.e. where a patient's body identifies the medical device as foreign and rejects it. Such responses can begin as early as on insertion of the medical device into the body, which can cause inflammation and trigger an immediate rush of inflammatory-mediating cells and proteins to the area of insertion. Proteins typically then non-specifically adsorb to the medical device surface, forming a protein layer which becomes a provisional matrix, through which cells and bacteria gathering in the area can identify and interact with the foreign body. Foreign body responses can ultimately cause numerous problems, including device clogging and infection. The negative impacts are often exacerbated when such medical devices are inserted by untrained personnel—e.g. by a user themselves in the absence of a medical professional. Furthermore, acute responses are particularly common for medical devices which are inserted subcutaneously or intravenously into the body, and these can have many harmful and even life-threatening consequences.

Medical device surface coatings and additives have been investigated to overcome the above issues. However, these have not been without disadvantages. Further, very few, if any, of these coatings and additives have been successful with fluoropolymer medical devices, especially PTFE medical devices. The chemical inertness of fluoropolymers makes them notoriously difficult to chemically modify or coat with an additive. Such polymers also display practical incompatibility with a vast range of chemistry commonly employed in medical device surface coatings and additives.

Recent efforts have looked to coating medical devices with phosphoester-based species. However, the phosphoester group is susceptible to hydrolysis, and such compounds are moisture sensitive and not easy to synthesize and handle. These traits render such approaches non-ideal, in particular for use with medical devices where long-term material stability is of crucial importance.

There exists a need for medical devices with further safeguards to ameliorate one or more of the above issues; in particular safeguards which are suitable for use with fluoropolymer-containing medical devices.

It is an aim of embodiments of the present invention to address or ameliorate one or more of the above problems of the prior art. In particular, it is an aim of embodiments of the present invention to provide a fluoropolymer-containing medical device which has one or more of the following advantages:
Excellent mechanical properties.
A non-stick and/or lubricious surface. Easy to insert and remove from the body.
Antibiofouling properties.
Protein-repellent properties/provides minimal protein adsorption.
Low risk of initiating foreign body responses or delayed foreign body response initiation.
Safe to use. No chemical leaching from the medical device, especially when inserted into the body.
Safe and effective even when implanted into the body for long periods of time.
Ease of manufacture.

It is also an aim of embodiments of the present invention to overcome or mitigate at least one problem of the prior art, whether expressly described herein or not.

### SUMMARY OF THE INVENTION

According to a first aspect of the invention, there is provided a medical device comprising a fluoropolymer surface comprising at least one species independently chosen from: a carboxybetaine, a sulfobetaine, and combinations and/or polymers thereof.

According to another aspect of the invention, there is provided a medical device comprising a fluoropolymer surface comprising at least one species independently selected from the group consisting of: a carboxybetaine, a sulfobetaine, and combinations and/or polymers thereof.

At least one carboxybetaine or sulfobetaine species on a fluoropolymer surface provides the surface of the medical device with a high level of resistance to protein adsorption and adhesion. The species protects the fluoropolymer surface from being targeted by foreign body responses. The species also displays high chemical compatibility with the fluoropolymer medical device surface; the fluoropolymer surface displays excellent mechanical properties and provides for a lubricious non-stick surface. Further, carboxybetaine and sulfobetaine species are chemically stable and not easily susceptible to degradation and hydrolysis.

In some preferred embodiments, there is provided a medical device comprising a fluoropolymer surface comprising at least one carboxybetaine and/or a polymer thereof.

In some embodiments, the species is present is present as a coating on the fluoropolymer surface. In some embodiments, at least 75% of the coating is the species, or at least 76, 77, 78, 79, 80, 81, 82, 83, 84, 85, 86, 87, 88, 89, 90, 91, 92, 93, 94, 95, 96, 97, 98, 99, or 100% of the coating is the species. In some embodiments, no greater than 95, 90, 85, or no greater than 80% of the coating is the species.

In some embodiments, the fluoropolymer is independently chosen from: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

In some embodiments, the fluoropolymer is independently selected from the group consisting of: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

The fluoropolymer may be independently chosen from: PTFE, fluorinated ethylene-propylene, polyvinylidene fluoride, and combinations, blends or copolymers thereof.

The fluoropolymer may be independently selected from the group consisting of: PTFE, fluorinated ethylene-propylene, polyvinylidene fluoride, and combinations, blends or copolymers thereof.

In a particularly preferred embodiment, the fluoropolymer is or comprises PTFE. PTFE provides excellent mechanical properties and demonstrates good compatibility with the species, despite its high fluorine-to-carbon ratio.

In some embodiments, at least one species comprises at least one carboxybetaine and/or at least one carboxybetaine polymer. A carboxybetaine refers to a zwitterionic species comprising a positively charged quaternary ammonium group and a negatively charged carboxylate group.

In some embodiments, at least one species comprises at least one sulfobetaine and/or at least one sulfobetaine polymer. A sulfobetaine refers to a zwitterionic species comprising a positively charged quaternary ammonium species and a negatively charged sulfonate group.

In some embodiments, at least one species comprises: at least one carboxybetaine and/or at least one carboxybetaine polymer; and at least one sulfobetaine and/or at least one sulfobetaine polymer.

In some embodiments, the fluoropolymer surface comprises 2 or at least 2 different carboxybetaine and/or sulfobetaine species, or 3 or at least 3, 4 or at least 4, 5 or at least 5, 6 or at least 6, 7 or at least 7, 8 or at least 8, 9 or at least 9, or 10 or at least 10 different species. In some embodiments, the fluoropolymer surface comprises no greater than 10 different species, or no greater than 9, 8, 7, 6, 5, 4, 3, or no greater than 2 different species. In some embodiments, the fluoropolymer comprises a single species.

At least one carboxybetaine and/or sulfobetaine species may preferably comprise at least one polymer. At least one polymer may comprise a homopolymer or copolymer. At least one homopolymer or copolymer may comprise an oligomer or polymer. At least one polymer may comprise a copolymer that is independently chosen from: a block copolymer, a random copolymer, an alternate copolymer, and combinations thereof. At least one polymer may comprise a copolymer that is independently selected from the group consisting of: a block copolymer, a random copolymer, an alternate copolymer, and combinations thereof. At least one carboxybetaine and/or sulfobetaine species may be independently chosen from: a linear polymer, a branched polymer, a graft polymer, a dendritic polymer, a star polymer, a dendronized polymer, a comb polymer, a polymer brush, a ladder polymer, and combinations thereof. At least one carboxybetaine and/or sulfobetaine species may be independently selected from the group consisting of: a linear polymer, a branched polymer, a graft polymer, a dendritic polymer, a star polymer, a dendronized polymer, a comb polymer, a polymer brush, a ladder polymer, and combinations thereof.

At least one species may comprise at least one poly(carboxybetaine) and/or at least one poly(sulfobetaine).

In some embodiments, at least one species comprises at least one poly(carboxybetaine). At least one poly(carboxybetaine) may comprise a polymer comprising at least one carboxybetaine repeat unit. At least one poly(carboxybetaine) may comprise a homopolymer, wherein substantially all repeat units of the polymer are carboxybetaine repeat units. At least one poly(carboxybetaine) may comprise a copolymer. At least one copolymer may contain at least one non-carboxybetaine repeat unit. Alternatively, all repeat units of at least one copolymer may be carboxybetaine repeat units.

In some embodiments, at least one species comprises at least one poly(carboxybetaine) composed of repeat units, wherein at least 25% of the total number of repeat units are carboxybetaine repeat units, or at least 50, 75, 80, 85, 90, 95, 96, 97, 98, or at least 99% of the total number of repeat units are carboxybetaine repeat units.

At least one poly(carboxybetaine) may comprise at least one repeat unit derived from a monomer containing a polymerizable group, preferably an unsaturated group. At least one poly(carboxybetaine) may comprise at least one repeat unit derived from a monomer that is independently chosen from: a carboxybetaine acrylate, a carboxybetaine alkacrylate, a carboxybetaine acrylamide, a carboxybetaine alkacrylamide, a carboxybetaine vinyl compound, a carboxybetaine epoxide, and combinations thereof. At least one poly(carboxybetaine) may comprise at least one repeat unit derived from a monomer that is independently selected from the group consisting of: a carboxybetaine acrylate, a carboxybetaine alkacrylate, a carboxybetaine acrylamide, a carboxybetaine alkacrylamide, a carboxybetaine vinyl compound, a carboxybetaine epoxide, and combinations thereof. At least one poly(carboxybetaine) may comprise at least one repeat unit derived from a monomer independently chosen from: a carboxybetaine acrylate, a carboxybetaine alkacrylate, a carboxybetaine acrylamide, a carboxybetaine alkacrylamide, and combinations thereof. At least one poly(carboxybetaine) may comprise at least one repeat unit derived from a monomer independently selected from the group consisting of: a carboxybetaine acrylate, a carboxybetaine alkacrylate, a carboxybetaine acrylamide, a carboxybetaine alkacrylamide, and combinations thereof. In some embodiments, at least one poly(carboxybetaine) may comprise at least one repeat unit derived from a monomer independently chosen from: a carboxybetaine acrylate, a carboxybetaine methacrylate, a carboxybetaine acrylamide, a carboxybetaine methacrylamide, and combinations thereof. In some embodiments, at least one poly(carboxybetaine) may comprise at least one repeat unit derived from a monomer independently selected from: a carboxybetaine acrylate, a carboxybetaine methacrylate, a carboxybetaine acrylamide, a carboxybetaine methacrylamide, and combinations thereof. In some preferred embodiments, at least one poly(carboxybetaine) comprises at least one acrylate and/or alkacrylate polymer.

In some embodiments, at least one poly(carboxybetaine) comprises at least one repeat unit derived from an unsaturated monomer of Formula (I):

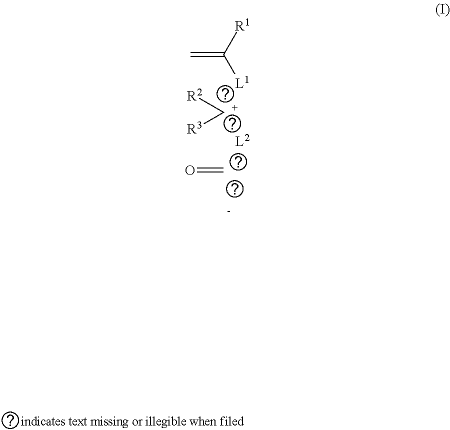

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a linker that covalently couples the ammonium group to the carboxylate group.

In some preferred embodiments, there is provided a medical device comprising a fluoropolymer surface comprising at least one species independently chosen from: a carboxybetaine, a sulfobetaine, and combinations and/or polymers thereof, wherein at least one species is a poly(carboxybetaine) comprising at least one repeat unit derived from an unsaturated monomer of Formula (I):

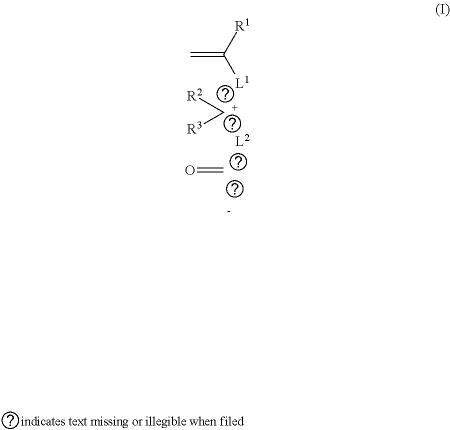

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a linker that covalently couples the ammonium group to the carboxylate group.

In some preferred embodiments, there is provided a medical device comprising a fluoropolymer surface comprising at least one species independently selected from the group consisting of: a carboxybetaine, a sulfobetaine, and combinations and/or polymers thereof, wherein at least one species is a poly(carboxybetaine) comprising at least one repeat unit derived from an unsaturated monomer of Formula (I):

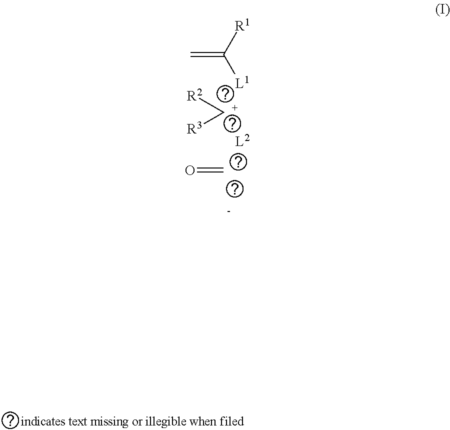

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a linker that covalently couples the ammonium group to the carboxylate group.

In some embodiments, R¹ is: H; or a straight or branched C1-C10 alkyl, preferably C1-C5 alkyl, more preferably C1-C3 alkyl. R¹ may be: H; or a straight-chain C1-C10 alkyl, preferably C1-C5 alkyl, more preferably C1-C3 alkyl. R¹ may be H. R¹ may be a straight or branched, preferably straight-chained, C1-C10 alkyl, preferably C1-C5 alkyl, more preferably C1-C3 alkyl. In some embodiments, R¹ is independently chosen from: methyl, ethyl, and propyl. In some embodiments, R¹ is independently selected from the group consisting of: methyl, ethyl, and propyl. In some preferred embodiments, R¹ is methyl.

In some embodiments, R² and R³ are the same or different, and are: a straight or branched, preferably straight-chained, C1-C10 alkyl, preferably C1-C5 alkyl, more preferably C1-C3 alkyl. In some embodiments, R² and R³ are the same or different, and are independently chosen from: methyl, ethyl, and propyl. In some embodiments, R² and R³ are the same or different, and are independently selected from the group consisting of: methyl, ethyl, and propyl. In some embodiments, at least one of R¹ and R² is methyl.

In some embodiments, R² and R³ are the same. R² and R³ may be the same and may be: a straight or branched, preferably straight-chained, C1-C10 alkyl, preferably C1-C5 alkyl, more preferably C1-C3 alkyl. In some embodiments, R² and R³ are the same, and are independently chosen from: methyl, ethyl, and propyl. In some embodiments, R² and R³ are the same, and are independently selected from the group consisting of: methyl, ethyl, and propyl. In some embodiments, both R¹ and R² are methyl.

L¹ may include a coupling functional group that couples the remainder of L¹ to the unsaturated polymerizable moiety. In some embodiments, the coupling functional group is an ester or an amide. L¹ may be: —C(═O)X—(CH₂)ₙ—; wherein X is: N or O; and n is: 1-10; preferably 1-5; more preferably 1-3; even more preferably 1-2; or 2. X may be N; and n may be: 1-10; preferably 1-5; more preferably 1-3; even more preferably 1-2; or 2. X may be 0; and n may be: 1-10; preferably 1-5; more preferably 1-3; even more preferably 1-2; or 2.

In some preferred embodiments, there is provided a medical device comprising a fluoropolymer surface comprising at least one species independently chosen from: a carboxybetaine, a sulfobetaine, and combinations and/or polymers thereof, wherein at least one species is a poly(carboxybetaine) comprising at least one repeat unit derived from an unsaturated monomer of Formula (I):

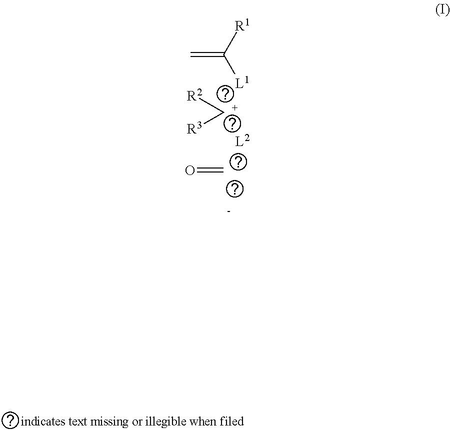

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
—C(═O)X—(CH₂)ₙ—; wherein X is: N or O; and n is: 1-5, preferably 1-3, and more preferably 1-2;
L² is:
a linker that covalently couples the ammonium group to the carboxylate group.

In some preferred embodiments, there is provided a medical device comprising a fluoropolymer surface comprising at least one species independently selected from the group consisting of: a carboxybetaine, a sulfobetaine, and combinations and/or polymers thereof, wherein at least one species is a poly(carboxybetaine) comprising at least one repeat unit derived from an unsaturated monomer of Formula (I):

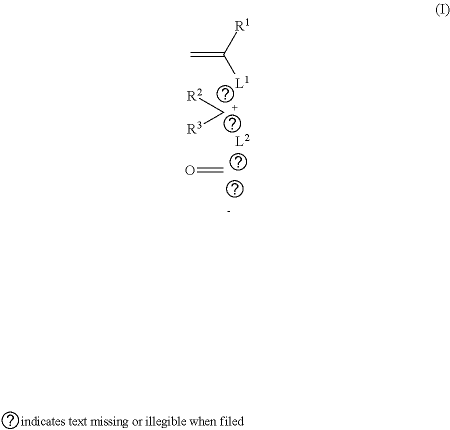

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
—C(═O)X—(CH₂)ₙ—; wherein X is: N or O; and n is: 1-5, preferably 1-3, and more preferably 1-2;
L² is:
a linker that covalently couples the ammonium group to the carboxylate group.

L² may be: a straight or branched, preferably straight-chained, C1-C10 alkyl, preferably C1-C5 alkyl, more preferably C1-C3 alkyl, even more preferably C1-C2 alkyl, or C2 alkyl.

In some preferred embodiments, there is provided a medical device comprising a fluoropolymer surface comprising at least one species independently chosen from: a carboxybetaine, a sulfobetaine, and combinations and/or polymers thereof, wherein at least one species is a poly(carboxybetaine) comprising at least one repeat unit derived from an unsaturated monomer of Formula (I):

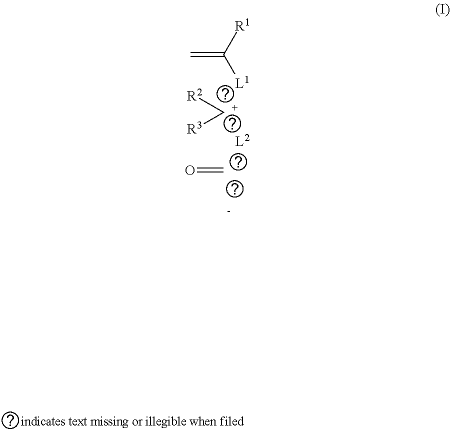

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a straight-chained C1-C5 alkyl, preferably C1-C3 alkyl, and more preferably C1-C2 alkyl.

In some preferred embodiments, there is provided a medical device comprising a fluoropolymer surface comprising at least one species independently selected from the group consisting of: a carboxybetaine, a sulfobetaine, and combinations and/or polymers thereof, wherein at least one species is a poly(carboxybetaine) comprising at least one repeat unit derived from an unsaturated monomer of Formula (I):

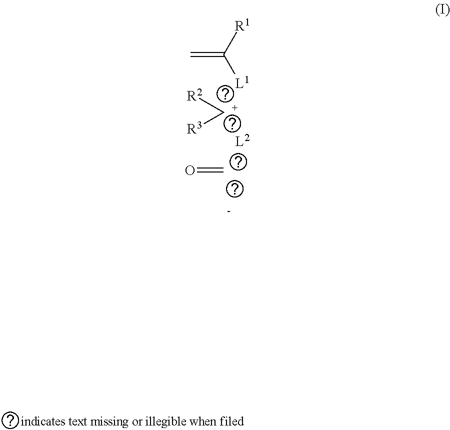

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a straight-chained C1-C5 alkyl, preferably C1-C3 alkyl, and more preferably C1-C2 alkyl.

In some embodiments, R¹ is: H; or a straight-chain C1-C10 alkyl, preferably C1-C5 alkyl, more preferably C1-C3 alkyl; and R² and R³ are the same and may be: a straight or branched, preferably straight-chained, C1-C10 alkyl, preferably C1-C5 alkyl, more preferably C1-C3 alkyl. In some embodiments, R¹ is independently chosen from: methyl, ethyl, and propyl; and R² and R³ are the same and are independently chosen from: methyl, ethyl, and propyl. In some embodiments, R¹ is independently selected from the group consisting of: methyl, ethyl, and propyl; and R² and R³ are the same and are independently selected from the group consisting of: methyl, ethyl, and propyl. In some embodiments, R¹, R² and R³ are the same. In some embodiments, R¹, R² and R³ are methyl.

In some preferred embodiments, there is provided a medical device comprising a fluoropolymer surface comprising at least one species independently chosen from: a carboxybetaine, a sulfobetaine, and combinations and/or polymers thereof, wherein at least one species is a poly(carboxybetaine) comprising at least one repeat unit derived from an unsaturated monomer of Formula (I):

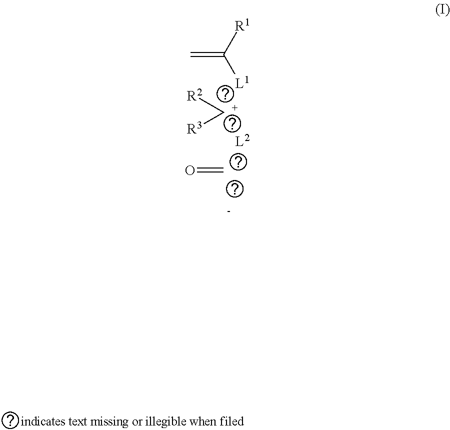

wherein: R¹, R² and R³ are the same, and preferably methyl;
L¹ is:
a linker that covalently couples the ammonium group to the unsaturated polymerizable moiety;
L² is:
a linker that covalently couples the ammonium group to the carboxylate group.

In some preferred embodiments, there is provided a medical device comprising a fluoropolymer surface comprising at least one species independently selected from the group consisting of: a carboxybetaine, a sulfobetaine, and combinations and/or polymers thereof, wherein at least one species is a poly(carboxybetaine) comprising at least one repeat unit derived from an unsaturated monomer of Formula (I):

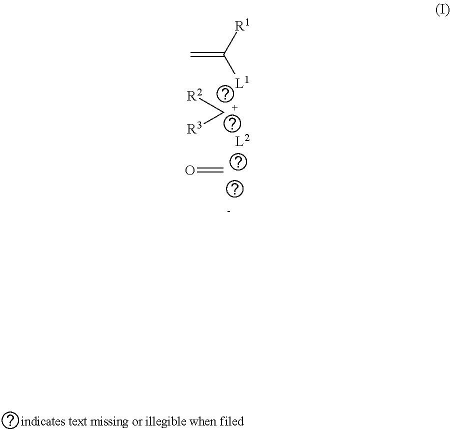

wherein: R¹, R² and R³ are the same, and preferably methyl;
L¹ is:
a linker that covalently couples the ammonium group to the unsaturated polymerizable moiety;
L² is:
a linker that covalently couples the ammonium group to the carboxylate group.

In some embodiments, R¹ is: H; or a straight-chain C1-C10 alkyl, preferably C1-C5 alkyl, more preferably C1-C3 alkyl; and L¹ is: —C(═O)X—(CH₂)ₙ—; wherein X is: N or O; and n is: 1-10; preferably 1-5; more preferably 1-3; even more preferably 1-2; or 2. In some embodiments, R¹ is independently chosen from: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2. In some embodiments, R¹ is independently selected from the group consisting of: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2.

In some embodiments, R¹ is: H; or a straight-chain C1-C10 alkyl, preferably C1-C5 alkyl, more preferably C1-C3 alkyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently chosen from: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently selected from the group consisting of: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, R² and R³ are the same and may be: a straight or branched, preferably straight-chained, C1-C10 alkyl, preferably C1-C5 alkyl, more preferably C1-C3 alkyl; and L¹ is: —C(═O)X—(CH₂)ₙ—; wherein X is: N or O; and n is: 1-10; preferably 1-5; more preferably 1-3; even more preferably 1-2; or 2. In some embodiments, R² and R³ are the same and are independently chosen from: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2.

In some embodiments, R² and R³ are the same and are independently selected from the group consisting of: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2.

In some embodiments, R² and R³ are the same and may be: a straight or branched, preferably straight-chained, C1-C10 alkyl, preferably C1-C5 alkyl, more preferably C1-C3 alkyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R² and R³ are the same and are independently chosen from: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R² and R³ are the same and are independently selected from the group consisting of: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, R¹ is: H; or a straight-chain C1-C10 alkyl, preferably C1-C5 alkyl, more preferably C1-C3 alkyl; R² and R³ are the same and may be: a straight or branched, preferably straight-chained, C1-C10 alkyl, preferably C1-C5 alkyl, more preferably C1-C3 alkyl; and L¹ is: —C(═O)X—(CH₂)ₙ—; wherein X is: N or O; and n is: 1-10; preferably 1-5; more preferably 1-3; even more preferably 1-2; or 2. In some embodiments, R¹ is independently chosen from: methyl, ethyl, and propyl; R² and R³ are the same and are independently chosen from: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2. In some embodiments, R¹ is independently selected from the group consisting of: methyl, ethyl, and propyl; R² and R³ are the same and are independently selected from the group consisting of: methyl, ethyl, and propyl; and L¹ is: —C(═O)X—(CH₂)ₙ—; wherein X is: N or O; and n is: 1-10; preferably 1-5; more preferably 1-3; even more preferably 1-2; or 2. In some embodiments, R¹, R² and R³ are the same; and L¹ is: —C(═O)X—(CH₂)ₙ—; wherein X is: N or O; and n is: 1-10; preferably 1-5; more preferably 1-3; even more preferably 1-2; or 2. In some embodiments, R¹, R² and R³ are methyl; and L¹ is: —C(═O)X—(CH₂)ₙ—; wherein X is: N or O; and n is: 1-10; preferably 1-5; more preferably 1-3; even more preferably 1-2; or 2.

In some embodiments, R¹ is: H; or a straight-chain C1-C10 alkyl, preferably C1-C5 alkyl, more preferably C1-C3 alkyl; R² and R³ are the same and may be: a straight or branched, preferably straight-chained, C1-C10 alkyl, preferably C1-C5 alkyl, more preferably C1-C3 alkyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently chosen from: methyl, ethyl, and propyl; R² and R³ are the same and are independently chosen from: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently selected from the group consisting of: methyl, ethyl, and propyl; R² and R³ are the same and are independently selected from the group consisting of: methyl, ethyl, and propyl; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹, R² and R³ are the same; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹, R² and R³ are methyl; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, R² and R³ are the same and may be: a straight or branched, preferably straight-chained, C1-C10 alkyl, preferably C1-C5 alkyl, more preferably C1-C3 alkyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R² and R³ are the same and are independently chosen from: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R² and R³ are the same and are independently selected from the group consisting of: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, R¹ is: H; or a straight-chain C1-C10 alkyl, preferably C1-C5 alkyl, more preferably C1-C3 alkyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently chosen from: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently selected from the group consisting of: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl.

In some embodiments, R¹ is: H; or a straight-chain C1-C10 alkyl, preferably C1-C5 alkyl, more preferably C1-C3 alkyl; R² and R³ are the same and may be: a straight or branched, preferably straight-chained, C1-C10 alkyl, preferably C1-C5 alkyl, more preferably C1-C3 alkyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently chosen from: methyl, ethyl, and propyl; R² and R³ are the same and are independently chosen from: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹ is independently selected from the group consisting of: methyl, ethyl, and propyl; R² and R³ are the same and are independently selected from the group consisting of: methyl, ethyl, and propyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹, R² and R³ are the same; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl. In some embodiments, R¹, R² and R³ are methyl; L¹ is: —C(═O)X—(CH₂)ₙ—; wherein X is: N or O; and n is: 1-10; preferably 1-5; more preferably 1-3; even more preferably 1-2; or 2; and L² is: a straight or branched, preferably straight-chained, C1-C10 alkyl, preferably C1-C5 alkyl, more preferably C1-C3 alkyl, even more preferably C1-C2 alkyl, or C2 alkyl.

In a particular embodiment, R¹, R² and R³ are methyl; L¹ is: —C(═O)O—(CH₂)₂—; and L¹ is a linear C2 alkyl. In such embodiments, at least one poly(carboxybetaine) comprises at least one repeat unit derived from 3-[[2-(Methacryloyloxy)ethyl]dimethylammonio]propionate.

In some embodiments, at least one species comprises at least one poly(sulfobetaine). At least one poly(sulfobetaine) may comprise a polymer comprising at least one sulfobetaine repeat unit. At least one poly(sulfobetaine) may comprise a homopolymer, wherein substantially all repeat units of the polymer are sulfobetaine repeat units. At least one poly(sulfobetaine) may comprise a copolymer. At least one copolymer may contain at least one non-sulfobetaine repeat unit. Alternatively, all repeat units of at least one copolymer may be sulfobetaine repeat units.

In some embodiments, at least one species comprises at least one poly(sulfobetaine) composed of repeat units, wherein at least 25% of the total number of repeat units are sulfobetaine repeat units, or at least 50, 75, 80, 85, 90, 95, 96, 97, 98, or at least 99% of the total number of repeat units are sulfobetaine repeat units.

At least one poly(sulfobetaine) may comprise at least one repeat unit derived from a monomer containing a polymerizable group, preferably an unsaturated group. At least one poly(sulfobetaine) may comprise at least one repeat unit derived from a monomer that is independently chosen from: a sulfobetaine acrylate, a sulfobetaine alkacrylate, a sulfobetaine acrylamide, a sulfobetaine alkacrylamide, a sulfobetaine vinyl compound, a sulfobetaine epoxide, and combinations thereof. At least one poly(sulfobetaine) may comprise at least one repeat unit derived from a monomer that is independently selected from the group consisting of: a sulfobetaine acrylate, a sulfobetaine alkacrylate, a sulfobetaine acrylamide, a sulfobetaine alkacrylamide, a sulfobetaine vinyl compound, a sulfobetaine epoxide, and combinations thereof. At least one poly(sulfobetaine) may comprise at least one repeat unit derived from a monomer independently chosen from: a sulfobetaine acrylate, a sulfobetaine alkacrylate, a sulfobetaine acrylamide, a sulfobetaine alkacrylamide, and combinations thereof. At least one poly(sulfobetaine) may comprise at least one repeat unit derived from a monomer independently selected from the group consisting of: a sulfobetaine acrylate, a sulfobetaine alkacrylate, a sulfobetaine acrylamide, a sulfobetaine alkacrylamide, and combinations thereof. In some embodiments, at least one poly(sulfobetaine) may comprise at least one repeat unit derived from a monomer independently chosen from: a sulfobetaine acrylate, a sulfobetaine methacrylate, a sulfobetaine acrylamide, a sulfobetaine methacrylamide, and combinations thereof. In some embodiments, at least one poly(sulfobetaine) may comprise at least one repeat unit derived from a monomer independently selected from: a sulfobetaine acrylate, a sulfobetaine methacrylate, a sulfobetaine acrylamide, a sulfobetaine methacrylamide, and combinations thereof. In some preferred embodiments, at least one poly(sulfobetaine) comprises at least one acrylate and/or alkacrylate polymer.

In some embodiments, at least one poly(sulfobetaine) comprises at least one repeat unit derived from an unsaturated monomer of Formula (II):

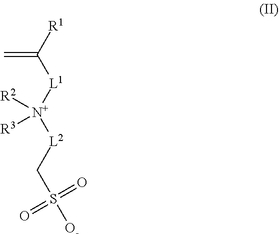

wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a linker that covalently couples the ammonium group to the sulfonate group.

Statements of invention above relating to any one or more of: R¹, R², R³, L¹, and L² from Formula (I) may also be applied mutatis mutandis to Formula (II).

In a particular embodiment, R¹, R² and R³ are methyl; L¹ is: —C(═O)O—(CH₂)₂—; and L² is a linear C2 alkyl. In such embodiments, at least one poly(sulfobetaine) comprises at least one repeat unit derived from 3-[[2-(Methacryloyloxy)ethyl]dimethylammonio]propane-1-sulfonate.

In some embodiments, at least one species comprises at least one poly(carboxybetaine) and at least one poly(sulfobetaine). At least poly(carboxybetaine) and/or at least one poly(sulfobetaine) may preferably be as described in statements of invention above. In such embodiments, at least one poly(carboxybetaine) and/or at least one poly(sulfobetaine) may be a homopolymer.

At least one species may comprise at least one copolymer comprising at least one carboxybetaine repeat unit and at least one sulfobetaine repeat unit. At least one carboxybetaine repeat unit and/or at least one sulfobetaine repeat unit may preferably be as described in statements of invention above. In some embodiments, at least one species comprises a copolymer comprising carboxybetaine and sulfobetaine repeat units in a carboxybetaine to sulfobetaine repeat unit ratio of between 1:10 to 10:1. At least one copolymer species may have carboxybetaine to sulfobetaine repeat unit ratio of between 1:10 to 1:1, or between 1:7.5 to 1:1, or between 1:5 to 1:1, or between 1:2.5 to 1:1. At least one copolymer species may have carboxybetaine to sulfobetaine repeat unit ratio of between 10:1 to 1:1, or between 7.5:1 to 1:1, or between 5:1 to 1:1, or between 2.5:1 to 1:1.

At least one species may comprise a polymer, which may be as described in statements of invention above, wherein the polymer has at least one repeat unit, or at least 2, 3, 4, 5, 6, 7, 8, 9, or at least 10 repeat units, or at least 20, 30, 40, 50, 75, 100, 150, 200, 250, 300, 350, 400, 450, 500, 750, 1000, 2000, 3000, 4000, 5000, 6000, 7000, 8000, 9000, or at least 10,000 repeat units. At least one species may comprise a polymer having no greater than 50,000 repeat units, or no greater than 45,000, 40,000, 35,000, or no greater than 30,000 repeat units.

At least one species may comprise a polymer, which may be as described in statements of invention above, the polymer having a molecular weight of at least 400 Da (Daltons), or at least 500, 600, 700, 800, 900, or at least 1,000 Da. At least one species may have a molecular weight of no greater than 1,000,000 Da, or no greater than 900,000, or no greater than 800,000, or no greater than 700,000, or no greater than 600,000, or no greater than 500,000, or no greater than 400,000, or no greater than 300,000, or no greater than 200,000 Da. At least one species may have a molecular weight of between 500-1,000,000 Da, or between 700-500,000 Da, or between 1,000-200,000 Da, or between 2,000-80,000 Da.

In some embodiments, at least one species is adsorbed to the fluoropolymer surface. At least one species may be physisorbed to the fluoropolymer surface. In preferred embodiments, at least one species may be chemisorbed to the fluoropolymer surface.

In some embodiments, at least one species is covalently bonded to the fluoropolymer surface. At least one species may be ionically and/or electrostatically bonded to the fluoropolymer surface. In some embodiments, at least one species is both covalently and ionically/electrostatically bonded to the fluoropolymer surface.

In some embodiments, at least one species is directly bonded to the fluoropolymer surface. At least one species may be bonded to the fluoropolymer surface via a linker. At least one species may be bonded to the linker by a covalent bonding method. At least one species may be bonded to the linker by an ionic and/or electrostatic bonding method. The fluoropolymer surface may be bonded to the linker by a covalent bonding method. The fluoropolymer surface may be bonded to the linker by an ionic and/or electrostatic bonding method. In some embodiments, at least one species is bonded to the linker by a covalent or ionic/electrostatic bonding method and the linker is bonded to the fluoropolymer surface by the same bonding method. Alternatively, at least one species may be bonded to the linker by a covalent or ionic/electrostatic bonding method and the linker may be bonded to the fluoropolymer surface by the opposite bonding method. In some embodiments, at least one species is bonded to the linker by a covalent bonding method and the linker is bonded to the fluoropolymer surface by a covalent or ionic/electrostatic bonding method. In some embodiments, at least one species is bonded to the linker by an ionic and/or electrostatic bonding method and the linker is bonded to the fluoropolymer surface by a covalent or ionic/electrostatic bonding method.

In preferred embodiments, the fluoropolymer surface is an activated fluoropolymer surface. Throughout this specification, the term “fluoropolymer surface” may be used to refer to an “activated fluoropolymer surface”. The activated fluoropolymer surface may comprise at least one electronegative atom. The fluoropolymer surface may be oxidised and may comprise at least one oxygen-containing moiety. In some embodiments, at least one species and/or linker is covalently bonded to the activated fluoropolymer surface via at least one oxygen-containing moiety on the fluoropolymer surface. At least one species and/or linker may be covalently bonded to the activated fluoropolymer surface through an ether and/or ester bond with at least one oxygen-containing moiety on the fluoropolymer surface.

At least one species and/or linker may be ionically and/or electrostatically bonded to the activated fluoropolymer surface. In some embodiments, at least one species and/or linker is bonded to the activated fluoropolymer surface via a hydrogen bonding interaction with at least one oxygen-containing moiety on the activated fluoropolymer surface. In such embodiments, the species and/or linker may act as a hydrogen bond donor. The activated fluoropolymer surface may act as a hydrogen bond acceptor.

In preferred embodiments, at least one species is bonded to the fluoropolymer surface and/or to a linker by a covalent bond. In some embodiments, at least one species is bonded to the fluoropolymer surface and/or a linker by a carbon-carbon bond, preferably a carbon-carbon polymer bond.

In some embodiments, at least one species is bonded to a linker by a covalent bond and the linker is bonded to the fluoropolymer surface by a covalent bond. In some embodiments, at least one species is bonded to a linker by a carbon-carbon covalent bond; and the linker is bonded to the fluoropolymer surface by a covalent bond with at least one oxygen-containing moiety on the fluoropolymer surface. At least one species may be bonded to the linker by a carbon-carbon covalent bond; and the linker may be bonded to the fluoropolymer surface through an ether and/or ester bond with at least one oxygen-containing moiety on the fluoropolymer surface, preferably through an ether bond with at least one oxygen-containing moiety on the fluoropolymer surface.

In some embodiments, the linker is derived from a linking compound comprising a bi- or poly-functional molecule comprising at least two reactive functional groups. A reactive functional group may be independently chosen from: a nucleophilic group, an electrophilic group, and a polymerizable moiety. A reactive functional group may be independently selected from: a nucleophilic group, an electrophilic group, and a polymerizable moiety. The linker may be derived from a linking compound comprising a polymerizable moiety, preferably an unsaturated group, such as a vinyl group. The linker may be derived from a linking compound comprising a polymerizable moiety and an electrophilic moiety. The linker may be derived from a linking compound comprising a polymerizable unsaturated group, preferably an acrylate or alkacrylate group, such as a methacrylate group. The electrophilic moiety may preferably comprise an electrophilic carbon centre. In some embodiments, the electrophilic carbon centre comprises a carbon atom bonded to an electronegative atom. The carbon atom may be bonded to an electronegative atom independently chosen from: a halogen and an oxygen. The carbon atom may be bonded to an electronegative atom independently selected from: a halogen and an oxygen. The electrophilic moiety may comprise an epoxide group. In embodiments, the linker may be derived from glycidyl acrylate and/or a glycidyl alkacrylate. In a particular embodiment, the linker is derived from glycidyl methacrylate.

In some embodiments, the species is present at a total concentration of at least 0.1, 0.2, 0.3, 0.4, or of at least 0.5 wt. % of the medical device. The species may be present at a total concentration of no greater than 20 wt. % of the medical device, or no greater than 15, 10, 5, 4, 3, 2, 1, 0.75 or of no greater than 0.5 wt % of the medical device. The species may be present at a total concentration of between 0.1-20 wt. %, or between 0.5-15 wt. % or 0.5-5 wt. % of the medical device.

In some preferred embodiments, there is provided a medical device comprising a fluoropolymer surface comprising at least one species independently chosen from: a carboxybetaine, a sulfobetaine, and combinations and/or polymers thereof, wherein the species is present at a total concentration of at least 0.5 wt. % of the medical device.

In some preferred embodiments, there is provided a medical device comprising a fluoropolymer surface comprising at least one species independently selected from the group consisting of: a carboxybetaine, a sulfobetaine, and combinations and/or polymers thereof, wherein the species is present at a total concentration of at least 0.5 wt. % of the medical device.

In some preferred embodiments, there is provided a medical device comprising a fluoropolymer surface comprising at least one species independently chosen from: a carboxybetaine, a sulfobetaine, and combinations and/or polymers thereof, wherein the species is present at a total concentration of between 0.5-15 wt. %, or between 0.5-5 wt. % of the medical device.

In some preferred embodiments, there is provided a medical device comprising a fluoropolymer surface comprising at least one species independently selected from the group consisting of: a carboxybetaine, a sulfobetaine, and combinations and/or polymers thereof, wherein the species is present at a total concentration of between 0.5-15 wt. %, or between 0.5-5 wt. % of the medical device.

The species is preferably present at and/or on the fluoropolymer surface. In some embodiments, the species is present at and/or on at least 5% of the total surface area of the fluoropolymer surface, or at least 10, 15, 20, 25, 30, 35, 40, 45, 50, 55, 60, 65, 70, 75, 80, 85, 90, 95, 96, 97, 98, or at least 99% of the total surface area of the fluoropolymer surface, preferably at least 75% or at least 90% of the total surface area of the fluoropolymer surface or between 75% and 100% of the total surface area of the fluoropolymer surface. In some embodiments, the species is present at and/or on no greater than 95% of the total surface area of the fluoropolymer surface, or no greater than 90, 85, or no greater than 80% of the total surface area of the fluoropolymer surface.

In some embodiments, the species comprises a layer that is on the fluoropolymer surface. In some embodiments, the species is adsorbed to the fluoropolymer surface to form the layer. The layer may be a monolayer. In some embodiments, the layer is a layer of coating comprising the species, which may be as described in statements of invention above.

In some embodiments, at least 75% of the layer comprising the species, or at least 76, 77, 78, 79, 80, 81, 82, 83, 84, 85, 86, 87, 88, 89, 90, 91, 92, 93, 94, 95, 96, 97, 98, 99, or 100% of the layer is the species. In some embodiments, no greater than 95, 90, 85, or no greater than 80% of the layer comprising the species is the species.

In some embodiments, the layer comprising the species has a thickness of at least 1 μm, or of at least 2, 3, 4, 5, 6, 7, 8, 9, 10, 15, 20, 25, 30, 35, 40, 45, or of at least 50 μm. The layer comprising the species may have a thickness of no more than 10000 μm, or of no more than 9000, 8000, 7000, 6000, 5000, 4000, 3000, 2000, 1000, 900, 800, 700, 600, 500, 400, or of no more than 300 μm.

In some embodiments, the species comprises an integral part of the fluoropolymer surface. The species may comprise an integral part of the fluoropolymer surface over at least 5% of the total area of the fluoropolymer surface, or at least 10, 15, 20, 25, 30, 35, 40, 45, 50, 55, 60, 65, 70, 75, 80, 85, 90, 95, 96, 97, 98, or at least 99% of the total area of the fluoropolymer surface, preferably at least 75% or at least 90% of the total area of the fluoropolymer surface or between 75% and 100% of the total area of the fluoropolymer surface. In some embodiments, the species forms an integral part of the fluoropolymer surface over no greater than 95% of the total area of the fluoropolymer surface, or no greater than 90, 85, or no greater than 80% of the total area of the fluoropolymer surface.

In some embodiments, the medical device comprises a tubular body comprising the fluoropolymer surface.

The fluoropolymer surface may be or comprise an outer and/or an inner surface of the tubular body. The fluoropolymer surface may preferably be or comprise an outer surface of the tubular body.

In some embodiments, the fluoropolymer surface comprises at least 5% of the outer surface area of the tubular body, or at least 10, 20, 30, 40, 50, 60, or preferably at least 70, or at least 80, 90, 95, 96, 97, 98, or at least 99% of the outer surface area of the tubular body, or 100% of the outer surface area of the tubular body. The fluoropolymer surface may comprise no greater than 95%, or no greater than 90, 85, or no greater than 80% of the outer surface area of the tubular body.

In some embodiments, the species is located at and/or on at least 50, 55, 60, 65, 70, 75, 80, 85, 90, 95, 96, 97, 98 or at least 99% of the outer surface area of the tubular body, preferably at least 75% or at least 90% of the outer surface area of the tubular body or between 75% and 100% of the outer surface area.

In preferred embodiments, the medical device is an insertable medical device. In some embodiments, the medical device is a cannula or a catheter, preferably which is configured to be inserted into a body. In preferred embodiments, at least one species is present at and/or on at least part of a surface of the cannula or catheter that is configured to be inserted into the body.

In some preferred embodiments, there is provided a medical device comprising a fluoropolymer surface comprising at least one species independently chosen from: a carboxybetaine, a sulfobetaine, and combinations and/or polymers thereof, wherein the medical device is a cannula or catheter.

In some preferred embodiments, there is provided a medical device comprising a fluoropolymer surface comprising at least one species independently selected from the group consisting of: a carboxybetaine, a sulfobetaine, and combinations and/or polymers thereof, wherein the medical device is a cannula or catheter.

In some embodiments, the cannula or catheter is independently chosen from: a urinary cannula or catheter, an intravenous cannula or catheter, a nasal cannula or catheter, and a microcannula.

In some embodiments, the cannula or catheter is independently selected from the group consisting of: a urinary cannula or catheter, an intravenous cannula or catheter, a nasal cannula or catheter, and a microcannula.

The cannula or catheter may be an indwelling (Foley) catheter or cannula. Such a cannula/catheter is typically inserted and kept in a body for long periods of time, such as several days to months. Alternatively, the cannula or catheter may be an intermittent catheter or cannula. Such a cannula/catheter is typically inserted into a body for short time periods, such as less than a day.

In preferred embodiments, the medical device is a cannula that is part of an infusion set. The cannula may be part of an infusion set comprising a body which comprises a fluid part. In some embodiments, the body of the infusion set is attachable to the body of a user, in use. The body of the infusion set may be attachable to the body of the user via an adhesive part, in use. The adhesive part may be attachable to skin, in use. The adhesive part may attach the body of the infusion set to the user's skin, in use. The fluid part may be connected to the body of the infusion set or comprise part of the body of the infusion set. The fluid part may provide a fluid path through the infusion set. The fluid part may allow for fluid communication between the body of the infusion set and the cannula. The cannula may be attached to the fluid part or directly to the body of the infusion set. An end of the cannula may preferably be insertable into the body of a user, in use. In some embodiments, the cannula comprises an insertion needle on an end thereof, which can help to insert the cannula into the body of the user. The infusion set may further comprise an inserter part to assist insertion of the cannula into the body of the user. The inserter part may be an automatic inserter part or a manual inserter part.

The infusion set may further comprise a pump. The pump may assist in transporting substances from the infusion set into the body of a user, and vice versa. In some embodiments, the pump is attached to the insertion set via a connector. The pump may be attached to the body of the infusion set via the connector. The connector may comprise a tube which may be attached to a hub which controls the pump.

In some embodiments, the medical device is a cannula that is part of a patch pump. The patch pump may comprise a patch that is attachable to the body of a user, in use. The patch may comprise an adhesive. The patch may be attachable to skin through the adhesive, in use. The patch may comprise a fluid part. The fluid part may provide a fluid path through the patch pump. The cannula may be attached to the fluid part. An end of the cannula may preferably be insertable into the body of a user, in use. In some embodiments, the cannula comprises an insertion needle on an end thereof, which can help to insert the cannula into the body of the user. The patch may further comprise a pump, which may be an integral part of the patch or may be attached thereto. The pump may assist in transporting substances from the patch pump into the body of a user, and vice versa.

In some preferred embodiments, there is provided a medical device comprising a fluoropolymer surface comprising at least one species independently chosen from: a carboxybetaine, a sulfobetaine, and combinations and/or polymers thereof, wherein the medical device is a cannula that is part of an infusion set or patch pump.

In some preferred embodiments, there is provided a medical device comprising a fluoropolymer surface comprising at least one species independently selected from the group consisting of: a carboxybetaine, a sulfobetaine, and combinations and/or polymers thereof, wherein the medical device is a cannula that is part of an infusion set or patch pump.

In some embodiments, the cannula is part of an infusion set or patch pump for the delivery of a substance into the body. The cannula may be part of an intravenous and/or subcutaneous infusion set or patch pump. The cannula may be part of an infusion set or patch pump for the subcutaneous delivery of a substance into the body, such as for the subcutaneous delivery on insulin into the body.

In some embodiments, the catheter or cannula comprises a hollow tubular body. The hollow tubular body may comprise an outer surface and/or an inner surface. The outer surface may comprise at least one of the group consisting of: an external facing surface of the body, a lumen of the body, and any eyelets present on the body. In preferred embodiments, the outer surface is the external-facing surface of the body and/or the inner lumen. In some embodiments, the outer surface may comprise the external-facing surface of the body, the inner lumen, and the eyelets. The inner surface of the body may comprise a lumen of the body.

In some embodiments, the species is present at and/or on an inner surface of the body, an outer surface of the body, or both. In preferred embodiments, the species is present at and/or on at least an outer surface of the body.

According to the second aspect of the invention, there is provided an infusion set or patch pump comprising a cannula comprising a fluoropolymer surface comprising at least one species independently chosen from: a carboxybetaine, a sulfobetaine, and combinations and/or polymers thereof.

According to another aspect of the invention, there is provided an infusion set or patch pump comprising a cannula comprising a fluoropolymer surface comprising at least one species independently selected from the group consisting of: a carboxybetaine, a sulfobetaine, and combinations and/or polymers thereof.

The cannula may preferably be a medical device of the first aspect of the invention. Statements of invention for the first aspect of the invention above may also be applied mutatis mutandis to the second aspect of the invention.

According to a third aspect of the invention, there is provided a method of manufacturing a medical device, the method comprising the steps of:
(a) Providing a medical device comprising a fluoropolymer surface;
(b) Activating the fluoropolymer surface; and
(c) Functionalising the activated fluoropolymer surface with at least one species independently chosen from: a carboxybetaine, a sulfobetaine, and combinations and/or polymers thereof.

According to another aspect of the invention, there is provided a method of manufacturing a medical device, the method comprising the steps of:
(a) Providing a medical device comprising a fluoropolymer surface;
(b) Activating the fluoropolymer surface; and
(c) Functionalising the activated fluoropolymer surface with at least one species independently selected from the group consisting of: a carboxybetaine, a sulfobetaine, and combinations and/or polymers thereof.

The medical device of the third aspect of the invention is preferably the medical device of the first aspect of the invention. Statements of invention above relating to the medical device of the first aspect of the invention or to any of its components may also be applied to the third aspect of the invention. Other statements of invention for the first and second aspects of the invention above may also be applied mutatis mutandis to the third aspect of the invention.

Statements of invention below relating to the third aspect of the invention may also be applied mutatis mutandis to the first and second aspects of the invention.

In some embodiments, step (a) comprises forming the medical device by a melt-extrusion or injection moulding procedure. The method may comprise melt-extruding or injection moulding a fluoropolymer to form a tubular body of the medical device. In some embodiments, the fluoropolymer is provided in granulate or powder form prior to melt-extrusion or injection-moulding.

In some embodiments, step (b) comprises introducing at least one reactive group on the fluoropolymer surface. Step (b) may comprise cleaving at least one polymer chain on the fluoropolymer surface, and introducing at least one reactive group on the surface. In some embodiments, at least one reactive group comprises at least one electronegative atom. In some embodiments, at least one reactive group may be independently chosen from: an oxygen-containing moiety, an unsaturated moiety, a radical, and combinations thereof. In some embodiments, at least one reactive group may be independently selected from the group consisting of: an oxygen-containing moiety, an unsaturated moiety, a radical, and combinations thereof.

Step (b) may comprise oxidising the fluoropolymer surface. In some embodiments, step (b) is performed under atmospheric oxygen conditions. In other embodiments, step (b) may be performed under an oxygen enriched atmosphere. Step (b) may produce an activated fluoropolymer surface comprising at least one oxygen-containing reactive moiety. At least one oxygen-containing moiety may be independently chosen from: a peroxy group, a hydroxy group, a carbonyl group, and derivatives and/or combinations thereof. At least one oxygen-containing moiety may be independently selected from the group consisting of: a peroxy group, a hydroxy group, a carbonyl group, and derivatives and/or combinations thereof. The carbonyl group may be independently chosen from: a carboxyl group, an aldehyde, a ketone, an acid fluoride, and combinations thereof. The carbonyl group may be independently selected from the group consisting of: a carboxyl group, an aldehyde, a ketone, an acid fluoride, and combinations thereof.

In some embodiments, step (b) comprises producing an activated fluoropolymer surface comprising at least one unsaturated reactive moiety.

In some preferred embodiments, the method of manufacturing a medical comprises the steps of:
(a) Providing a medical device comprising a fluoropolymer surface;
(b) Activating the fluoropolymer surface to produce an activated fluoropolymer surface comprising at least one unsaturated reactive moiety; and
(c) Functionalising the activated fluoropolymer surface with at least one species independently chosen from: a carboxybetaine, a sulfobetaine, and combinations and/or polymers thereof.

In some preferred embodiments, the method of manufacturing a medical comprises the steps of:
(a) Providing a medical device comprising a fluoropolymer surface;
(b) Activating the fluoropolymer surface to produce an activated fluoropolymer surface comprising at least one unsaturated reactive moiety; and
(c) Functionalising the activated fluoropolymer surface with at least one species independently selected from the group consisting of: a carboxybetaine, a sulfobetaine, and combinations and/or polymers thereof.

At least one unsaturated reactive moiety may be independently chosen from: an alkene, an alkyne, and derivatives and/or combinations thereof. At least one unsaturated reactive moiety may be independently selected from the group consisting of: an alkene, an alkyne, and derivatives and/or combinations thereof. Such unsaturated reactive moieties may react via polymerisation-type reactions. In any of the embodiments described herein in which polymerisation is performed, any suitable polymerisation process may be used, such as conventional condensation, addition or free radical graft polymerization (FRGP) or controlled radical polymerization (CRP), such as ATRGP, RAFT and NMGP.

In some embodiments, step (b) comprises the step of activating the fluoropolymer surface across at least 5% of the total area of the fluoropolymer surface, or at least 10, 20, 30, 40, 50, 60, 70, 80, 90, 95, 96, 97, 98, or across at least 99% of the total area of the fluoropolymer surface, or 100% of the total area of the fluoropolymer surface. Step (b) may comprise the step of activating the fluoropolymer surface across no greater than 95% of the total area of the fluoropolymer surface, or across no greater than 90, 85, or no greater than 80% of the total area of the fluoropolymer surface.

Step (b) may comprise defluorinating or partially defluorinating the fluoropolymer surface. Step (b) may comprise defluorinating at least 5% of the fluoropolymer surface, or at least 10, 20, 30, 40, 50, 60, 70, 80, 90, 95, 96, 97, 98, or at least 99% of the fluoropolymer surface, or 100% of the fluoropolymer surface. Step (b) may comprise defluorinating no greater than 95% of the fluoropolymer surface, or no greater than 90, 85, or no greater than 80% of the fluoropolymer surface.

Step (b) may comprise reducing the average fluorine-to-carbon atomic ratio (F/C ratio) of the fluoropolymer surface to a value of no greater than 1.2, or no greater than 1, 0.9, 0.8, 0.7, 0.6, 0.5, 0.4, 0.3, 0.2, or no greater than 0.1.

Step (b) may comprise increasing the average surface energy of the fluoropolymer surface to a value of at least 25 mN/m, or at least 30, 35, 40, 45, 50, 55, 60, or at least 65 mN/m.

Step (b) may comprise reducing the average contact angle of the fluoropolymer surface to a value of no greater than 80°, or no greater than 70, 60, 50, 40, or no greater than 30°.

Step (b) may comprise activating the fluoropolymer surface with at least one fluoropolymer surface activation method independently chosen from: plasma treatment, treatment with a reducing agent, corona discharge treatment, ion beam treatment, laser treatment, and combinations thereof. Step (b) may comprise activating the fluoropolymer surface with at least one fluoropolymer surface activation method independently selected from the group consisting of: plasma treatment, treatment with a reducing agent, corona discharge treatment, ion beam treatment, laser treatment, and combinations thereof.

In some embodiments, step (b) comprises the step of plasma treating the fluoropolymer surface. Plasma treating the fluoropolymer surface may comprise applying a plasma stream to the fluoropolymer surface. The fluoropolymer surface may be directly contacted with plasma as it is generated, or in a separate post-plasma area. If the surface is directly contacted with plasma during generation, this may take place in a plasma reactor. By post-plasma area, it is meant in the present disclosure an area out of the plasma, located downstream of a plasma forming gas flow introduced in the plasma wherein reactive species such as radicals are still present. That post-plasma area is particularly useful for delicate substrate surfaces such as polymers.

Step (b) may comprise treating the fluoropolymer surface with a gaseous plasma. The plasma may comprise at least one plasma gas independently chosen from: hydrogen, oxygen, nitrogen, air, ammonia, argon, helium, carbon dioxide, water, methane, ethane, propane, butane, and any mixture thereof. The plasma may comprise at least one plasma gas independently selected from the group consisting of: hydrogen, oxygen, nitrogen, air, ammonia, argon, helium, carbon dioxide, water, methane, ethane, propane, butane, and any mixture thereof. The plasma gas may be carried by a carrier gas, which may be the same as the plasma gas or may be different to the plasma gas. In some embodiments, the carrier gas is an inert gas, such as argon, for example.

In some embodiments, the method may comprise treating the fluoropolymer surface with a primary gas and a secondary gas. The primary gas may be independently chosen from: hydrogen, oxygen, nitrogen, air, ammonia, argon, helium, carbon dioxide, water, methane, ethane, propane, butane, and any mixture thereof. The primary gas may be independently selected from the group consisting of: hydrogen, oxygen, nitrogen, air, ammonia, argon, helium, carbon dioxide, water, methane, ethane, propane, butane, and any mixture thereof. The primary gas may comprise an inert gas, which may comprise a noble gas. The primary gas may be independently chosen from: helium, argon, and combinations thereof. The primary gas may be independently selected from the group consisting of: helium, argon, and combinations thereof. The secondary gas may be independently chosen from: hydrogen, oxygen, nitrogen, air, ammonia, argon, helium, carbon dioxide, water, methane, ethane, propane, butane, and any mixture thereof. The secondary gas may be independently selected from the group consisting of: hydrogen, oxygen, nitrogen, air, ammonia, argon, helium, carbon dioxide, water, methane, ethane, propane, butane, and any mixture thereof. In some embodiments, the secondary gas is or comprises oxygen.

In some embodiments, the method may comprise treating the fluoropolymer surface with at least one plasma gas having a flow rate of at least 3 Lpm, or at least 6, 9, 12, or at least 15 Lpm. The method may comprise treating the fluoropolymer surface with at least one plasma gas having a flow rate of no greater than 50 Lpm, or no greater than 45, 40, 35, 30, 25, or of no greater than 20 Lpm. The method may comprise treating the fluoropolymer surface with at least one plasma gas having a flow rate of between 5-30 Lpm, or between 10-25, or between 15-20 Lpm. At least one plasma gas having such a flow rate may preferably be a primary gas.

In some embodiments, the method may comprise treating the fluoropolymer surface with at least one plasma gas having a flow rate of at least 0.025 Lpm, or at least 0.05, 0.075, 0.1, 0.2, 0.3, 0.4, 0.5, or at least 0.6 Lpm. The method may comprise treating the fluoropolymer surface with at least one plasma gas having a flow rate of no greater than 5 Lpm, or no greater than 4, 3, 2, 1, 0.9, 0.8, or no greater than 0.7 Lpm. The method may comprise treating the fluoropolymer surface with at least one plasma gas having a flow rate of between 0.025-1 Lpm, or of between 0.05-0.9, 0.075-0.8, 0.1-0.7, or between 0.15-0.65 Lpm. At least one plasma gas having such a flow rate may preferably be a secondary gas.

The step of plasma treating the fluoropolymer surface may introduce at least one reactive group on the fluoropolymer surface, preferably at least one oxygen-containing reactive moiety. Plasma treating the fluoropolymer surface may oxidise the fluoropolymer surface. In some embodiments, plasma treating the fluoropolymer surface is performed under atmospheric oxygen conditions. In other embodiments, plasma treating the fluoropolymer surface may be performed under an oxygen enriched atmosphere.

In some embodiments, the plasma treatment step uses cold plasma.

Cold plasma, otherwise known as non-thermal or non-equilibrium plasma, is the term used for cold temperature plasma formation at atmospheric pressures. Cold plasma is a plasma which is not in thermodynamic equilibrium, because the electron temperature is much hotter than the temperature of heavy species (ions and neutrals) in the plasma. Cold plasma is created when a sufficient amount of energy, higher than the ionization energy, is added to gaseous atoms and/or molecules, causing ionization and subsequently generating free electrons, photons, free radicals and ionic species. This excitation energy supplied to a gas to form a cold plasma can originate from electrical discharges, direct currents, radio frequencies, microwaves or other forms of electromagnetic radiation.

Non-limiting examples of cold plasma technologies and methodologies for generating cold plasma include atmospheric pressure plasma jet, dielectric barrier discharge, direct current (DC) glow discharge, electrical discharge plasma, microwave discharge, pulsed power discharge, radiofrequency (RF) discharge, and the like.

In some embodiments, the cold plasma is cold atmospheric plasma. The cold plasma may be an atmospheric pressure discharge cold plasma.

The temperature of the cold plasma may be at least 5° C. or at least 10° C. The temperature of the cold plasma may be no more than 60° or no more than 50° C. In some embodiments the cold plasma is at ambient temperature, such as between 15° C. and 35° C., for example.

The plasma or cold plasma may be at a pressure of between around 50 kPa and 150 kPa, preferably between around 60 kPa and 140 kPa, between around 70 kPa and 130 kPa, or between around 80 kPa and 120 kPa. In some embodiments the pressure may be between around 100 kPa and 103 kPa.

In other embodiments, the plasma or cold plasma may be applied under reduced pressure such as below 50 kPa, such as between 0.01 kPa and 40 kPa, or between 0.1 kPa and 25 kPa.

The plasma or cold plasma treatment may be performed at a radio-frequency (RF) power of at least 1 W, 5 W, 10 W, 15 W or at least 20 W. The plasma or cold plasma treatment may be performed at an RF power of no more than 2000 W, 1500 W, 1000 W, 500 W, 400 W, 300 W, 200 W, 100 W, 90 W, 80 W, 70 W or no more than 60 W. In some embodiments the treatment may be performed at an RF power of about 20 to 60 W. The treatment may be performed at an RF power of between 20-500 W, or between 30-450, 40-400, 50-350, 60-300, 70-250, 80-200, or between 90-170, or between 100-160 W.

The plasma or cold plasma treatment may be performed for a total time of at least 1 second, 2 seconds, 3 seconds, 4 seconds, 5 seconds or at least 10 seconds. The plasma or cold plasma treatment may be performed for no more than 600 seconds, 550 seconds, 500, 450, 400, or no more than 350 seconds. In some embodiments, the treatment may be performed for about 5 to 500 seconds, or for about 10-400 seconds, or for about 15-300 seconds.

The plasma or cold plasma treatment may be performed at a temperature of at least 5° C., or at least 10, 20, 30, 40, 50, or at least 60° C. The plasma or cold plasma treatment may be performed at a temperature of no greater than 200° C., or no greater than 180, 160, 140, 120, 100, 80, or no greater than 60° C. The plasma or cold plasma treatment may be performed at a temperature of between 20-100° C., or between 30-90, 40-80, 50-70, or between 55-65° C.

The plasma or cold plasma treatment may be performed at an RF power of between about 10 W to about 60 W, for a period of between about 5 seconds to about 120 seconds; and in some embodiments may be performed using the aforesaid RF and time ranges using a precursor gas chosen from hydrogen, oxygen, nitrogen, argon or helium.

The plasma or cold plasma treatment may be performed at an RF power of between about 10 W to about 60 W, for a period of between about 5 seconds to about 120 seconds; and in some embodiments may be performed using the aforesaid RF and time ranges using a precursor gas selected from the group consisting of hydrogen, oxygen, nitrogen, argon or helium.

In some embodiments, step (b) comprises treating the fluoropolymer surface with at least one reducing agent.

In some preferred embodiments, the method of manufacturing a medical comprises the steps of:
(a) Providing a medical device comprising a fluoropolymer surface;
(b) Activating the fluoropolymer surface by treating the surface with at least one reducing agent; and
(c) Functionalising the activated fluoropolymer surface with at least one species independently chosen from: a carboxybetaine, a sulfobetaine, and combinations and/or polymers thereof.

In some preferred embodiments, the method of manufacturing a medical comprises the steps of:
(a) Providing a medical device comprising a fluoropolymer surface;
(b) Activating the fluoropolymer surface by treating the surface with at least one reducing agent; and
(c) Functionalising the activated fluoropolymer surface with at least one species independently selected from the group consisting of: a carboxybetaine, a sulfobetaine, and combinations and/or polymers thereof.

The reducing agent may act to transfer electrons to the fluoropolymer surface. Such a method is particularly effective at producing a highly reactive fluoropolymer surface which can be easily functionalised with the carboxybetaine and/or sulfobetaine species. However, the method has no long-term implications on the stability of the modified surface—the method may in fact aid stability of the modified surface through surface crosslinking interactions generated on treatment with a reducing agent.

Treating the fluoropolymer surface with at least one reducing agent may generate at least one surface reactive group. In some embodiments, at least one reactive group may be as described in statements above and may be independently chosen from: an oxygen-containing moiety, an unsaturated moiety, a radical, and combinations thereof. In some embodiments, at least one reactive group may be as described in statements above and may be independently selected from the group consisting of: an oxygen-containing moiety, an unsaturated moiety, a radical, and combinations thereof.

The method may comprise the steps of: transferring at least one electron from the reducing agent to the fluoropolymer surface to generate a negatively charged or partially negatively charged surface group; and removing at least one fluorine from the surface group to generate a neutral defluorinated surface group. Fluorine may be removed from the surface group as fluoride or a derivative thereof. In some embodiments, the above steps may produce a radical-containing neutral defluorinated surface group. The above steps may be repeated to generate a non-radical neutral defluorinated surface group. The non-radical neutral defluorinated surface group may comprise a reactive group, preferably an unsaturated moiety, such as an alkene. The reactive group may participate in step (c) of the method of the third aspect of the invention. In some embodiments, the above steps may not be repeated, and the radical-containing neutral defluorinated surface group may participate directly in step (c) of the method of the third aspect of the invention.

At least one reducing agent used in step (b) of the invention may be independently chosen from: an alkali metal, an alkaline earth metal, a group III metal, a transition metal, and combinations thereof.

At least one reducing agent used in step (b) of the invention may be independently selected from the group consisting of: an alkali metal, an alkaline earth metal, a group III metal, a transition metal, and combinations thereof.

In preferred embodiments, at least one reducing agent comprises an alkali metal and/or an alkaline earth metal. At least one reducing agent may preferably comprise an alkali metal. At least one reducing agent may comprise an alkali metal independently chosen from: lithium, potassium, sodium, and combinations thereof. At least one reducing agent may comprise an alkali metal independently selected from the group consisting of: lithium, potassium, sodium, and combinations thereof. In a particularly preferred embodiment, at least one reducing agent comprises sodium.

In some embodiments, at least one reducing agent may be used with a stabilising species. The stabilising species may complex the reducing agent, preferably in the form of a salt. The stabilising species may accept an electron from the reducing agent, preferably to form a radical anion. The stabilising species may preferably be an aromatic compound. The stabilising species may be a polycyclic aromatic compound. The stabilising species may be independently chosen from: benzene, naphthalene, biphenyl, anthracene, pyrene, acenaphthylene, perylene, and derivatives thereof. The stabilising species may be independently selected from the group consisting of: benzene, naphthalene, biphenyl, anthracene, pyrene, acenaphthylene, perylene, and derivatives thereof. The stabilising species may preferably be naphthalene or a derivative thereof. In preferred embodiments, at least one reducing agent comprises an alkali metal and a naphthalene stabilising species which forms an alkali metal naphthalide, preferably sodium naphthalide.

At least one reducing agent may be provided as a solution. At least one reducing agent may be dissolved in a carrier solvent to provide the solution. The carrier solvent may comprise an aprotic solvent. The carrier solvent may comprise an ether, preferably an aprotic ether. In some embodiments, the carrier solvent comprises a glycol ether, preferably an aprotic glycol ether, such as a dialkyl glycol ether.

In some preferred embodiments, the method of manufacturing a medical comprises the steps of:
(a) Providing a medical device comprising a fluoropolymer surface;
(b) Activating the fluoropolymer surface by treating the surface with at least one reducing agent, wherein the reducing agent is dissolved in a carrier solvent comprising a glycol ether, preferably an aprotic glycol ether; and
(c) Functionalising the activated fluoropolymer surface with at least one species independently chosen from: a carboxybetaine, a sulfobetaine, and combinations and/or polymers thereof.

In some preferred embodiments, the method of manufacturing a medical comprises the steps of:
(a) Providing a medical device comprising a fluoropolymer surface;
(b) Activating the fluoropolymer surface by treating the surface with at least one reducing agent, wherein the reducing agent is dissolved in a carrier solvent comprising a glycol ether, preferably an aprotic glycol ether; and
(c) Functionalising the activated fluoropolymer surface with at least one species independently selected from the group consisting of: a carboxybetaine, a sulfobetaine, and combinations and/or polymers thereof.

In preferred embodiments, the carrier solvent is independently chosen from: monoglyme, diglyme, tetraglyme, and combinations thereof. In preferred embodiments, the carrier solvent is independently selected from the group consisting of: monoglyme, diglyme, tetraglyme, and combinations thereof. In a particularly preferred embodiment, the carrier solvent comprises diglyme.

Particularly preferably, the reducing agent comprises an alkali metal, preferably sodium and the carrier solvent comprises an aprotic glycol ether, preferably a dialkyl glycol ether, more preferably diglyme.

Such solvents enable high temperature etching, which accelerates and reduces the length of the surface treatment process.

In some embodiments, step (b) comprises treating the fluoropolymer surface with at least one reducing agent at a temperature of at least 5° C., or at least 10, 15, 20, 25, 30, 35, 40, or at least 45° C. Step (b) may comprise treating the fluoropolymer surface with at least one reducing agent at a temperature of no greater than 500° C., or no greater than 450, 400, 350, 300, 250, 200, 150, 100, 90, 80, 70, 60, or no greater than 50° C. Step (b) may comprise treating the fluoropolymer surface with at least one reducing agent at a temperature of between 5-100° C., or between 10-95, 20-90, 25-85, 30-80, 35-75, 40-70, 45-65, or between 50-65° C.

In some preferred embodiments, the method of manufacturing a medical comprises the steps of:
(a) Providing a medical device comprising a fluoropolymer surface;
(b) Activating the fluoropolymer surface by treating the surface with at least one reducing agent at a temperature of between 45-65° C., or between 50-65° C.; and
(c) Functionalising the activated fluoropolymer surface with at least one species independently chosen from: a carboxybetaine, a sulfobetaine, and combinations and/or polymers thereof.

In some preferred embodiments, the method of manufacturing a medical comprises the steps of:
(a) Providing a medical device comprising a fluoropolymer surface;
(b) Activating the fluoropolymer surface by treating the surface with at least one reducing agent at a temperature of between 45-65° C., or between 50-65° C.; and
(c) Functionalising the activated fluoropolymer surface with at least one species independently selected from the group consisting of: a carboxybetaine, a sulfobetaine, and combinations and/or polymers thereof.

Step (b) may comprise treating the fluoropolymer surface with at least one reducing agent at a temperature of between 10-70° C., or between 15-65, or between 20-60° C.

Such temperatures allow more active reducing agent to be released. Reducing agent viscosity is also reduced which allows for wetting of high aspect ratio features of the medical device.

In some embodiments, step (b) comprises treating the fluoropolymer surface with at least one reducing agent at a temperature of between 30-80° C., or between 35-75, 40-70, 45-65, or between 50-60° C.; and wherein the reducing agent is dissolved in a glycol ether carrier solvent, preferably an aprotic glycol ether solvent, more preferably a dialkyl glycol ether.

Step (b) may comprise treating the fluoropolymer surface with the reducing agent for at least 1 second, or at least 2, 3, 4, 5, 10, 15, 20, 25, or at least 30 seconds. Step (b) may comprise treating the fluoropolymer surface with the reducing agent for no greater than 300 seconds, or no greater than 280, 260, 240, 220, 200, 180, 160, 140, 120, 100, 80, or no greater than 60 seconds. Step (b) may comprise treating the fluoropolymer surface with the reducing agent for between 5-180 seconds, or between 10-160, 15-140, 20-120, 25-110, 30-100, 35-90, 40-80, 50-70, or between 55-65 seconds. Step (b) may comprise treating the fluoropolymer surface with the reducing agent for between 5-55 seconds, or between 10-50, 15-45, 20-40, or between 25-35 seconds.

Step (b) may comprise applying the reducing agent to the fluoropolymer surface, preferably as a solution. Step (b) may comprise submerging the medical device or the fluoropolymer surface in the solution.

Step (c) may comprise treating the activated fluoropolymer surface with at least one species, preferably as described for the first aspect of the invention. Step (c) may comprise treating the activated fluoropolymer surface with monomer units of at least one polymer species, preferably as described for the first aspect of the invention above.

Step (c) may comprise treating the surface with a solution of the species or monomers thereof in a solvent. The solvent may be a polar solvent, preferably a polar protic solvent. The solvent may comprise water. Alternatively, the solution may comprise an organic solvent, which may be a polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. The organic solvent may be or comprise an alcohol, which may be a C1-C10 alcohol, preferably C1-C5, more preferably C1-C3 alcohol. The alcohol may be independently chosen from: methanol, ethanol, propanol, isopropanol, and combinations thereof. The alcohol may be independently selected from the group consisting of: methanol, ethanol, propanol, isopropanol, and combinations thereof. The alcohol may be independently chosen from: methanol, ethanol, and combinations thereof. The alcohol may be independently selected from: methanol, ethanol, and combinations thereof. The species or monomers thereof may be present in the solution at a total concentration of at least 0.05 wt. %, or at least 0.1, 0.2, 0.3, 0.4, 0.5, 0.6, 0.7, 0.8, 0.9, or at least 1 wt. %. The species or monomers thereof may be present in the solution at a total concentration of no greater than 10 wt. %, or no greater than 9, 8, 7, 6, 5, 4, 3, 2, or no greater than 1 wt. %. The species or monomers thereof may be present in the solution at a total concentration of between 0.05-5 wt %, or between 0.1-2 wt. %, or between 0.5-1.5, or between 0.75-1.25 wt. %. The solution of the species or monomers thereof may further comprise a polymerisation initiator, preferably a free radical initiator. The radical initiator may comprise a peroxide. The peroxide may be chosen from: benzoyl peroxide (BPO), di-tert-butyl peroxide, cumene hydroperoxide, tert-butyl hydroperoxide, 2,5-bis(tert-butylperoxy)-2,5-dimethylhexane (DHBP), di(tert-butylperoxyisopropyl)benzene, dicumyl peroxide (DCP), 2,5-di(tert-butylperoxy)-2,5-dimethyl-3-hexyne (DTBPHY) or combinations and/or derivatives thereof. The peroxide may be selected from the group consisting of: benzoyl peroxide (BPO), di-tert-butyl peroxide, cumene hydroperoxide, tert-butyl hydroperoxide, 2,5-bis(tert-butylperoxy)-2,5-dimethylhexane (DHBP), di(tert-butylperoxyisopropyl)benzene, dicumyl peroxide (DCP), 2,5-di(tert-butylperoxy)-2,5-dimethyl-3-hexyne (DTBPHY) or combinations and/or derivatives thereof. The radical initiator may comprise an azo compound. The azo compound may be chosen from: AIBN, AMBN, ADVN, ACVA, dimethyl 2,2′-azobis(2-methylpropionate), AAPH, and 2,2′-azobis [2-(2-imidazolin-2-yl)-propane]dihydrochloride, or combinations and/or derivatives thereof. The azo compound may be selected from the group consisting of: AIBN, AMBN, ADVN, ACVA, dimethyl 2,2′-azobis(2-methylpropionate), AAPH, and 2,2′-azobis [2-(2-imidazolin-2-yl)-propane]dihydrochloride, or combinations and/or derivatives thereof. The radical initiator may comprise a photo-radical initiator, which may be chosen from: camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis(2-chlorophenyl)-4,4′,5,5′-tetraphenyl-1,2′-biimidazole, diphenyl(2,4,6-trimethylbenzoyl)phosphine oxide, phenylbis(2,4,6-trimethylbenzoyl)phosphine oxide, and lithium phenyl(2,4,6-trimethylbenzoyl)phosphinate, or combinations and/or derivatives thereof. The radical initiator may comprise a photo-radical initiator, which may be selected from the group consisting of: camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis(2-chlorophenyl)-4,4′,5,5′-tetraphenyl-1,2′-biimidazole, diphenyl(2,4,6-trimethylbenzoyl)phosphine oxide, phenylbis(2,4,6-trimethylbenzoyl)phosphine oxide, and lithium phenyl(2,4,6-trimethylbenzoyl)phosphinate, or combinations and/or derivatives thereof. The polymerisation initiator may be present in the solution at a total concentration of at least 0.05 wt. %, or at least 0.1, 0.2, 0.3, 0.4, 0.5, 0.6, 0.7, 0.8, 0.9, or at least 1, 1.25, 1.5, 1.75, or at least 2 wt. %. The polymerisation initiator may be present in the solution at a total concentration of no greater than 10 wt. %, or no greater than 9, 8, 7, 6, 5, 4, 3, or no greater than 2 wt. %. The polymerisation initiator may be present in the solution at a total concentration of between 0.1-5 wt. %, or between 0.5-4, 1-3, 1.5-2.5, or between 1.75-2.25 wt. %. In some embodiments, the polymerisation initiator may be present in the solution at a greater total concentration than the total concentration of the species or monomers thereof. The polymerisation initiator may be present in the solution at a total concentration of at least 1.1 times the total concentration of the species or monomers thereof, or at least 1.2, 1.3, 1.4, 1.5, 1.6, 1.7, 1.8, 1.9, or at least 2 times the total concentration of the species or monomers thereof.

In some embodiments, the solution of the species or monomers thereof comprises a polymerisation initiator and the solution is held for a time period before treating the activated fluoropolymer surface with the solution. The solution may be held for at least 5 minutes prior to treatment, or at least 10, 15, 20, 25, or at least 30 minutes prior to treatment. The solution may be held for no greater than 120 minutes prior to treatment, or no greater than 110, 100, 90, 80, 70, or no greater than 60 minutes prior to treatment. The solution may be held prior to treatment for between 5-85 minutes, 10-80, 20-70, or between 30-60 minutes. The solution may be held prior to treatment at a temperature of at least 5° C. or at least 10, 15 or at least 20° C. The solution may be held prior to treatment at a temperature of no greater than 100° C., or no greater than 90, 80, 70, 60, or no greater than 50° C. The solution may be held prior to treatment at a temperature of between 5-95° C., or between 10-90, 15-85, 20-80, 25-75, 30-70, 35-65, 40-60, or between 45-55° C.

Step (c) may comprise treating the surface with at least one species or monomers thereof for a total time of at least 5 minutes, or at least 10, 20, 30, 40, 50, 60, 70, 80, 90, 100, 110, 120, 130, 140, 150, 160, 170, 180, 190, 200, 210, 220, 230, or at least 240 minutes. Step (c) may comprise treating the surface with at least one species or monomers thereof for total time of no greater than 10 hours, or no greater than 9, 8, 7, 6, 5, 4.5, or no greater than 4 hours. Step (c) may comprise treating the surface with at least one species or monomers thereof for total time of between 0.25-5 hours, or between 0.5-4, or between 0.5-3, or between 0.5-2 hours.

Step (c) may comprise treating the surface with at least one species or monomers thereof at a temperature of at least 5° C. or at least 10, 15 or at least 20° C. Step (c) may comprise treating the surface with at least one species or monomers thereof at a temperature of no greater than 100° C., or no greater than 90, 80, 70, 60, or no greater than 50° C. Step (c) may comprise treating the surface with at least one species or monomers thereof at a temperature of between 5-95° C., or between 10-90, 15-85, 20-80, 25-75, 30-70, 35-65, 40-60, or between 45-55° C.

Step (c) may comprise grafting at least one polymeric species to the activated surface. The method may alternatively comprise polymerising at least one species from the activated surface, preferably by attaching a carboxybetaine or sulfobetaine species monomer to the surface and polymerising from the monomer to provide a polymeric species attached to the fluoropolymer surface.

The step of functionalising the activated fluoropolymer surface with at least one species may comprise bonding the species to the activated surface through a linker. The linker may be derived from a linking compound, preferably as described for the first aspect of the invention. The method may comprise the further step of treating the activated fluoropolymer surface with a linking compound. The method may comprise the step of first bonding the linking compound to the activated fluoropolymer surface, and then bonding at least one species or a monomer thereof to the linking compound. The method may comprise the step of treating the activated surface with the linking compound, optionally in the absence or presence of the species or monomers thereof; and then treating the surface with at least one species or monomers thereof. In some embodiments, the method comprises functionalising the activated surface with the linking compound to form a layer of the linking compound attached to the fluoropolymer surface.

The method may comprise treating the fluoropolymer surface with the linking compound for a total time of at least 5 minutes, or at least 10, 20, 30, 40, 50, or at least 60 minutes. The method may comprise treating the surface with the linking compound for a total time of no greater than 300 minutes, or no greater than 250, 200, or no greater than 150 minutes. The method may comprise treating the surface with the linking compound for a total time of between 20-100 minutes, or between 30-90, 40-80, 50-70, or between 55-65 minutes.

The method may comprise treating the surface with the linking compound at a temperature of at least 5° C. or at least 10, 15 or at least 20° C. The method may comprise treating the surface with the linking compound at a temperature of no greater than 100° C., or no greater than 90, 80, 70, 60, 50, 40, or no greater than 30° C. The method may comprise treating the surface with the linking compound at a temperature of between 5-45° C., or between 10-40, 15-35, or between 20-30° C.

The linking compound may be present neat or as a solution of the linking compound in a solvent. The solvent may be a polar solvent or non-polar solvent. The solvent may be an aprotic solvent. In some embodiments, the solution may be an aqueous solution. Alternatively, the solution may comprise an organic solvent, which may be a polar or non-polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. In some embodiments, the solvent is or comprises an ether, which may be a C1-C20 ether, preferably C1-C10 ether. The ether may be an alkyl tert-butyl ether, which may be independently chosen from: methyl tert-butyl ether, ethyl tert-butyl ether, propyl tert-butyl ether, and combinations thereof. The ether may be an alkyl tert-butyl ether, which may be independently selected from the group consisting of: methyl tert-butyl ether, ethyl tert-butyl ether, propyl tert-butyl ether, and combinations thereof. The linking compound may be present in the solution at a total concentration of at least 0.05 wt. %, or at least 0.1, 0.2, 0.3, 0.4, 0.5, 0.6, 0.7, 0.8, 0.9, 1, 1.5, or at least 2 wt. %. The linking compound may be present in the solution at a total concentration of no greater than 10 wt. %, or no greater than 9, 8, 7, 6, 5, 4, 3, or no greater than 2 wt. %. The linking compound may be present in the solution at a total concentration of between 0.05-10 wt %, or between 0.1-5 wt. %, or between 0.5-4, or between 1-3, or between 1.5-2.5 wt %.

At least one species or monomer thereof may be provided as a chemically modified derivative of the species. The chemically modified derivative may comprise at least one reactive group, preferably to facilitate attachment of the species to the fluoropolymer surface or linking compound. The reactive group may comprise a polymerizable moiety, preferably an unsaturated moiety. The unsaturated moiety may preferably comprise an acrylate or alkacrylate moiety, such as methacrylate. At least one species or monomer thereof may preferably be as described in statements of invention for the first aspect of the invention above. In some embodiments, the method comprises polymerising at least one species or monomer thereof through the polymerizable moiety. The method may comprise polymerising a polymeric species to produce a polymer comprising macromonomers.

In any of the embodiments described herein in which polymerisation is performed, any suitable polymerisation process may be used, such as conventional condensation, addition or free radical graft polymerization (FRGP) or controlled radical polymerization (CRP), such as ATRGP, RAFT and NMGP.

In some embodiments, the fluoropolymer surface is functionalised with at least one species via a free radical polymerisation method. The free radical polymerisation method may comprise a controlled/living free radical polymerisation method. These techniques are known to a skilled person in the art and employ the principle of an equilibrium between free radicals and various types of dormant species (depending on the specific type of polymerisation technique employed).

The controlled/living radical polymerisation techniques include nitroxide-mediated polymerisation, reversible addition fragmentation transfer polymerisation (RAFT) and atom transfer radical polymerisation (ATRP).

More detailed descriptions of polymerisation mechanisms and related chemistry is discussed for nitroxide-mediated polymerisation (Chapter 10, pages 463 to 522), ATRP (Chapter 11, pages 523 to 628) and RAFT (Chapter 12, pages 629 to 690) in the Handbook of Radical Polymerization, edited by Krzysztof Matyjaszewski and Thomas P. Davis, 2002, published by John Wiley and Sons Inc.

The controlled/living polymerisation processes leave a residue of reagent on the polymer chain such as (nitroxyl group from nitroxide-mediated), or a halogen from ATRP, thiocarbonylthio group from RAFT.

In some embodiments it is desirable to remove the residue i.e., remove the nitroxyl, halogen or thiocarbonylthio group. Processes are known to a skilled person to remove such groups, and the disclosure in EP2791184 provides a solution to remove thiocarbonylthio groups. Other such techniques are described, for example, in (Chong et at, Macromolecules 2007, 40, 4446-4455; Chong et al, Aust. J. Chem. 2006, 59, 755-762; Postma et al, Macromolecules 2005, 38, 5371-5374; Moad et al, Polymer International 60, no. 1, 2011, 9-25; and Wilcock et al, Polym. Chem., 2010, 1, 149-157.

In ATRP polymerisation, groups that may be transferred by a radical mechanism include halogens (from a halogen-containing compound) or various ligands. A more detailed review of groups that may be transferred is described in U.S. Pat. No. 6,391,996.

Examples of a halogen-containing compound that may be used in ATRP polymerisation include benzyl halides such as p-chloromethylstyrene, α-dichloroxylene, α,α-dichloroxylene, α,α-dibromoxylene, hexakis(α-bromomethyl)benzene, benzyl chloride, benzyl bromide, 1-bromo-1-phenylethane and 1-chloro-1-phenylethane; carboxylic acid derivatives which are halogenated at the α-position, such as propyl 2-bromopropionate, methyl 2-chloropropionate, ethyl 2-chloropropionate, methyl 2-bromopropionate, and ethyl 2-bromoisobutyrate; tosyl halides such as p-toluenesulfonyl chloride; alkyl halides such as tetrachloromethane, tribromomethane, I-vinylethyl chloride, and 1-vinylethyl bromide; and halogen derivatives of phosphoric acid esters, such as dimethylphosphoric acid.

In one embodiment when the halogen compound is employed, a transition metal such as copper is also present. The transition metal may be in the form of a salt. The transition metal is capable of forming a metal-to-ligand bond and the ratio of ligand to metal depends on the dentate number of the ligand and the co-ordination number of the metal. The ligand may be a nitrogen or phosphorus-containing ligand.

Examples of a suitable ligand include triphenylphosphine, 2,2-bipyridine, alkyl-2,2-bipyridine, such as 4,4-di-(5-heptyl)-2,2-bipyridine, tris(2-aminoethyl)amine (TREN), N,N,N′,N′,N″-pentamethyldiethylenetnamine, 4,4-do-(5-nonyl)-2,2-bipyridine, 1,1,4,7,10,10-hexamethyltriethylenetetramine and/or tetramethylethylenediamine. Further suitable ligands are described in, for example, International Patent application WO 97/47661. The ligands may be used individually or as a mixture. In one embodiment the nitrogen containing ligand is employed in the presence of copper. In one embodiment the ligand is phosphorus-containing with triphenyl phosphine (PPh₃) a common ligand. A suitable transition metal for a triphenyl phosphine ligand includes Rh, Ru, Fe, Re, Ni or Pd.

In RAFT polymerisation, a chain transfer agent may be used. A more detailed review of suitable chain transfer agents RAFT polymerisation, as described in International Patent Publication Nos. WO 98/01478, WO 99/31144 and WO 10/83569, is a polymerisation technique that exhibits characteristics associated with living polymerisation.

Examples of a suitable RAFT chain transfer agent include benzyl 1-(2-pyrrolidinone)carbodithioate, benzyl(1,2-benzenedicarboximido) carbodithioate, 2-cyanoprop-2-yl 1-pyrrolecarbodithioate, 2-cyanobut-2-yl 1-pyrrolecarbodithioate, benzyl 1-imidazolecarbodithioate, N,N-dimethyl-S-(2-cyanoprop-2-yl)dithiocarbamate, N,N-diethyl-S-benzyl dithiocarbamate, cyanomethyl 1-(2-pyrrolidone) carbodithoate, cumyl dithiobenzoate, 2-dodecylsuilphanylthiocarbonylsulphanyl-2-methyl-propionic acid butyl ester, O-phenyl-S-benzyl xanthate, N,N-diethyl S-(2-ethoxy-carbonylprop-2-yl)dithiocarbamate, dithiobenzoic acid, 4-chlorodithiobenzoic acid, O-ethyl-S-(1-phenylethyl)xanthtate, O-ethyl-S-(2-(ethoxycarbonyl)prop-2-yl)xanthate, O-ethyl-S-(2-cyanoprop-2-yl)xanthate, O-ethyl-S-(2-cyanoprop-2-yl)xanthate, O-ethyl-S-cyanomethyl xanthate, O-pentafluorophenyl-S-benzyl xanthate, 3-benzylthio-5,5-dimethylcyclohex-2-ene-1-thione or benzyl 3,3-di(benzylthio)prop-2-enedithioate, S,S′-bis-(α,α′-disubstituted-α″-acetic acid)-trithiocarbonate, S,S′-bis-(α,α′-disubstituted-α″-acetic acid)-trithiocarbonate or S-alkyl-S′-(α,α′-disubstituted-α″-acetic acid)-trithiocarbonates, benzyl dithiobenzoate, 1-phenylethyl dithiobenzoate, 2-phenylprop-2-yl dithiobenzoate, 1-acetoxyethyl dithiobenzoate, hexakis(thiobenzoylthiomethyl)benzene, 4-bis(thiobenzoylthiomethyli)benzene, 1,2,4,5-tetrakis(thiobenzoylthiomethyl)benzene, 1,4-bis-(2-(thiobenzoylthio)-prop-2-yl)benzene, 1-(4-methoxyphenyl)ethyl dithiobenzoate, benzyl dithioacetate, ethoxycarbonylmethyl dithioacetate, 2-(ethoxycarbonyl)prop-2-yl dithiobenzoate, 2,4,4-trimethylpent-2-yl dithiobenzoate, 2-(4-chlorophenyl)prop-2-yl dithiobenzoate, 3-vinylbenzyl dithiobenzoate, 4-vinylbenzyl dithiobenzoate, S-benzyl diethoxyphosphinyldithioformate, tert-butyl trithioperbenzoate, 2-phenylprop-2-yl 4-chlorodithiobenzoate, 2-phenylprop-2-yl 1-dithionaphthalate, 4-cyanopentanoic acid dithiobenzoate, dibenzyl tetrathioterephthalate, dibenzyl trithiocarbonate, carboxymethyl dithiobenzoate or poly(ethylene oxide) with dithiobenzoate end group or mixtures thereof.

In one embodiment a suitable RAFT chain transfer agent includes 2-Dodecylsulfanylthiocarbonylsulfanyl-2-methyl-propionic acid butyl ester, cumyl dithiobenzoate or mixtures thereof.

A discussion of the polymer mechanism of RAFT polymerisation is shown on page 664 to 665 in section 12.4.4 of Matyjaszewski et al.

In some embodiments, the fluoropolymer surface is functionalised with at least one species via an ionic polymerisation method, which may be an anionic polymerisation method. In such embodiments, at least one carboxybetaine and/or sulfobetaine species monomer may be functionalised with an epoxide group; said group being able to participate in an anionic polymerisation process. The ionic polymerisation method may comprise a controlled/living ionic polymerisation method, preferably an anionic polymerisation method.

When the polymeric species is prepared by anionic polymerisation techniques, initiators include, for example, hydrocarbyllithium initiators such as alkyllithium compounds (e.g. methyl lithium, n-butyl lithium, see-butyl lithium), cycloalkyllithium compounds (e.g. cyclohexyl lithium and aryl lithium compounds (e.g., phenyl lithium, 1-methylstyryl lithium, p-tolyl lithium, naphyl lithium and 1,1-diphenyl-3-methylpentyl lithium. Also, useful initiators include naphthalene sodium, 1,4-disodio-1,1,4,4-tetraphenylbutane, diphenylmethyl potassium or diphenylmethylsodium.

The ionic polymerisation process may be carried out in the absence of moisture and oxygen and in the presence of at least one inert solvent. In one embodiment anionic polymerisation is conducted in the absence of any impurity which is detrimental to an anionic catalyst system.

The inert solvent may include a hydrocarbon, an aromatic solvent or ether. Suitable solvents include isobutane, pentane, cyclohexane, benzene, toluene, xylene, tetrahydrofuran, diglyme, tetraglyme, orthoterphenyl, biphenyl, decalin or tetralin.

The ionic polymerisation process may be carried out at a temperature of 0° C. to −78° C.

A more detailed description of process to prepare polymers from an anionic process is discussed in Textbook of Polymer Science, edited by Fred W, Billmeyer Jr., Third Edition, 1984, Chapter 4, pages 88-90.

In some embodiments, steps (b) and (c) are performed simultaneously. In other embodiments, step (c) may be performed subsequently to step (b).

In some embodiments, the activation step is performed in the presence of at least one species. In other embodiments, the activation step is performed in the absence of the species, preferably prior to addition of the species.

In embodiments in which at least one species is bonded to the fluoropolymer surface through a linker, the method may comprise the step of functionalising the fluoropolymer surface with the linking compound simultaneously or subsequently to step (b). In some embodiments, the activation step is performed in the presence of the linking compound. In other embodiments, the activation step is performed in the absence of the linking compound, preferably prior to addition of the linking compound.

In embodiments in which step (b) comprises plasma treating the fluoropolymer surface, the method may comprise the step of subjecting the fluoropolymer surface to a first plasma jet in the presence of a linking compound to form a layer of the linking compound on the fluoropolymer surface. The method may comprise the further step of subjecting the linking compound layer to a second plasma jet in the presence of at least one species to attach the species to the linking compound layer.

In some embodiments, the method comprises a further step of sonicating the fluoropolymer surface. The sonication step may be performed at one or more of the following times: after step (b), after treating the fluoropolymer surface with a linking compound and before treating the surface with at least one species, and at the end of step (c), and any combination thereof. The or each sonication step may be performed for between 1-30 minutes, or between 5-20 minutes, or between 5-15 minutes. The sonication step may be performed in a polar solvent, which may be a polar protic solvent. The solvent may be an aqueous solvent and may be water.

The method may comprise a further step of washing the fluoropolymer surface. The washing step may be performed at one or more of the following times: after step (b), after treating the fluoropolymer surface with a linking compound and before treating the surface with at least one species, and at the end of step (c), and any combination thereof. The surface may be washed with a solvent, which may be a polar solvent. The solvent may be a polar protic solvent. The solvent may comprise an alcohol and/or water. The washing step may be performed at a temperature of between 20-120° C., or between 40-100, or between 60-80° C. The washing step may comprise a first washing step at ambient temperature and a second washing step at a temperature range independently selected from the above range. The first washing step may be performed with an organic solvent, preferably a polar organic solvent. The polar organic solvent may comprise a polar protic solvent, such as an alcohol. The second step may be performed with an aqueous solution or with water, preferably with deionised water.

According to a fourth aspect of the invention, there is provided the use of at least one species that is independently chosen from: a carboxybetaine, a sulfobetaine, and combinations and/or polymers thereof, as a protein-repellent in and/or on a medical device.

According to another aspect of the invention, there is provided the use of at least one species that is independently selected from the group consisting of: a carboxybetaine, a sulfobetaine, and combinations and/or polymers thereof, as a protein-repellent in and/or on a medical device.

According to a fifth aspect of the invention, there is provided a method of delivering a substance to or removing a substance from the body of a subject, the method comprising the steps of:
(a) Inserting a medical device comprising a fluoropolymer surface comprising at least one species independently chosen from: a carboxybetaine, a sulfobetaine, and combinations and/or polymers thereof into the body; and
(b) Delivering a substance to or removing a substance from the body via the medical device.

According to another aspect of the invention, there is provided a method of delivering a substance to or removing a substance from the body of a subject, the method comprising the steps of:
(a) Inserting a medical device comprising a fluoropolymer surface comprising at least one species independently selected from the group consisting of: a carboxybetaine, a sulfobetaine, and combinations and/or polymers thereof into the body; and
(b) Delivering a substance to or removing a substance from the body via the medical device.

The medical device may be a catheter or cannula, preferably as described for the first aspect of the invention. The medical device may be a cannula that is part of an infusion set or patch pump.

The method may comprise inserting the medical device into the body intravenously and/or subcutaneously.

The substance may be a drug. In some embodiments, the substance comprises insulin. Step (b) of the method may comprise delivering insulin to the body via the medical device.

The following statements apply to the fourth and fifth aspects of the invention.

The species and/or medical device may preferably be the species and medical device of the first aspect of the invention. Statements of invention for the first, second and third aspects of the invention above may also be applied mutatis mutandis to the fourth and fifth aspects of the invention.

### DETAILED DESCRIPTION OF THE INVENTION

In order that the invention may be more clearly understood embodiments thereof will now be described, by way of example only, with reference to the accompanying drawings, of which:

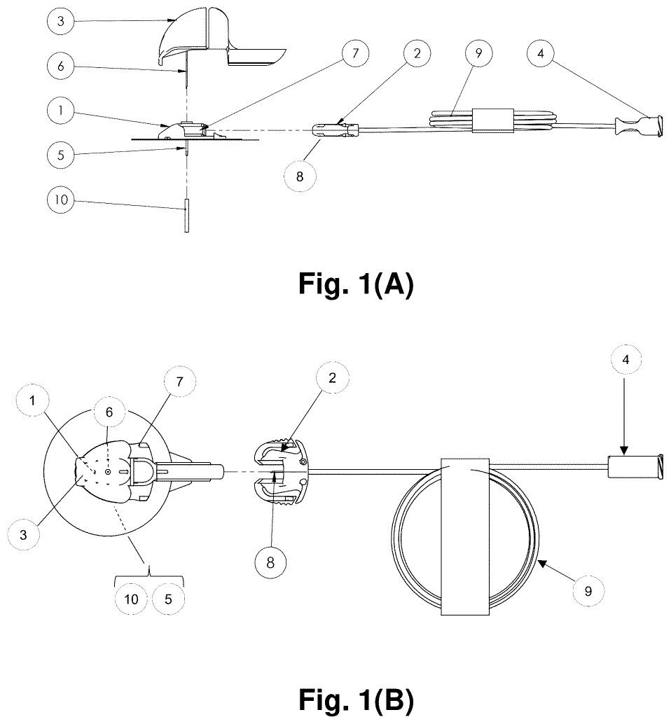

FIG. 1 shows (A) an exploded side-on view; and (B) a top-down view of an infusion set of the second aspect of the invention. Dashed lines represent points of connection of the components of the infusion set.

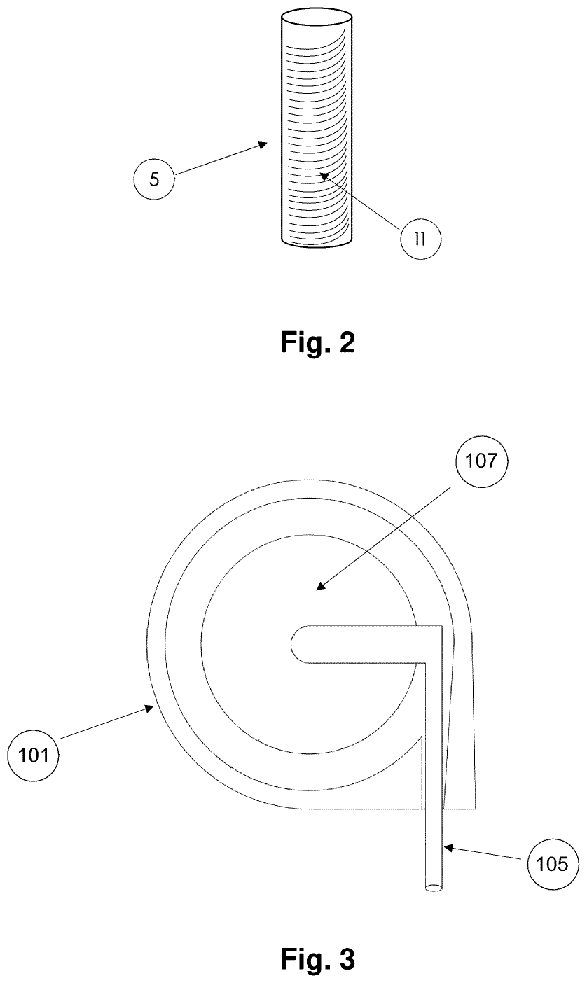

FIG. 2 shows an expanded side-on view of the cannula (5) as displayed in FIG. 1(A).

FIG. 3 shows a cross-sectional view of a patch pump of the second aspect of the invention.

### EXAMPLE 1

A first embodiment of a medical device of the first aspect of the invention is provided by a cannula containing a polymeric tubular body having a PTFE outer surface. The PTFE outer surface is functionalised with a carboxybetaine polymer derived from 3-[[2-(Methacryloyloxy)ethyl]dimethylammonio]propionate monomer units, which is bonded to the PTFE surface via a linker derived from glycidyl methacrylate.

The cannula is part of an infusion set of the second aspect of the invention for the subcutaneous delivery of insulin. Diagrams of the infusion set are displayed in FIGS. 1(A) and 1(B). With reference to the figures, the infusion set comprises a body (1), which is attachable to the skin of a user via an adhesive part of the body (1). The infusion set comprises the cannula (5) which extends from and projects away from the body (1) of the infusion set in the same direction that the adhesive part of the body (1) faces.

The body (1) comprises a fluid part (7), which is part of the body and provides a fluid path through the infusion set, allowing for fluid communication between the body (1) and the cannula (5). The fluid part (7) also contains a cartridge of insulin (not shown) for subcutaneous delivery. The arrangement also allows for fluid communication between the inside of the insulin cartridge and the cannula (5). The fluid part (7) is connected to a pump (not shown) via tubing (9). The fluid part (7) is connected to the tubing (9) at one end thereof via a connector needle (8) of a set connector (2). The other end of the tubing (9) contains a pump connector (4) through which the tubing (9) is attached to the pump.

The fluid part (7) and body (1) contain a channel extending therethrough which is aligned with the cannula (5). Such an arrangement allows for an insertion needle (6) to be passed through the channel and into the cannula (5), with the insertion needle (6) projecting in the same direction as the cannula (5) and extending out of the free, distal end of the cannula (5). An inserter (3) is connected to the insertion needle (6) and the needle (6) extends from the inserter (3). The infusion set further includes a needle cover (10) in which the insertion needle (6) is sheathed before use.

In use, the body (1) of the infusion set is attached to the skin of the user via the adhesive part of the body (1). The free end of the cannula (5), which projects from the body (1), is inserted into the body of the user with assistance from the insertion needle (6), which is inserted using the inserter (3) through the channel extending through the fluid part (7) and body (1) and through the cannula (5). The insertion needle (6) contacts the skin of the user and is inserted into the body of the user before the cannula (5), making insertion of the cannula (5) easier.

On insertion of the cannula (5) into the body of the user, the fluid part (7) is connected to the pump as described above. The insulin is delivered subcutaneously from the infusion set via the cannula (5), with assistance from the pump.

FIG. 2 shows an expanded side-on view of the cannula (5), as displayed in FIG. 1(A). FIG. 2 displays the carboxybetaine polymer as a layer or coating (11), which is formed on the PTFE outer surface of the cannula by bonding of the polymer to the PTFE.

### Cannula Preparation Method

The functionalised cannula was prepared as follows.

Surface Activation: a solution of sodium naphthalide in diglyme was preheated to 60° C. for 1 hour. The solution was then shaken vigorously for 2-3 seconds, after which the cannula was submerged in the solution for 30 seconds. The cannula was thereafter removed and immediately rinsed with isopropyl alcohol for 10 seconds. The cannula was then further rinsed with 70° C. deionised water for 15 seconds. The cannula was then left to air dry overnight.

XPS data showed that the fluorine-to-carbon atom ratio had changed from 2:1 (before surface activation) to 1:6 (after activation). XPS data also showed the presence of C—O bonds on the treated surface.

Linker functionalisation: the cannula was thereafter submerged in a solution of glycidyl methacrylate (2 wt % in methyl tert-butyl ether) at room temperature for 1 hour. The cannula was then removed, rinsed with deionised water at ambient temperature, and sonicated for 10 minutes in fresh deionised water. The cannula was then air dried.

Carboxybetaine functionalisation: a solution containing 1 wt % of 3-[[2-(Methacryloyloxy)ethyl]dimethylammonio]propionate and 2 wt % of AIBN polymerisation initiator was prepared in an ethanol solvent. The prepared solution was held at 50° C. for 30-60 minutes prior to use to initiate polymerisation. The cannula was submerged in the prepared solution at 50° C. for 1 hour, prior to rinsing with water, sonicating and air drying, as performed previously. Submerging the cannula in the solution allowed for covalent linkage of carboxybetaine polymer chains to the linker derived from glycidyl methacrylate which was present on the fluoropolymer surface.

### Results

The final functionalised cannula contained a thin layer of carboxybetaine polymer adsorbed to the fluoropolymer surface via a linker.

A protein adsorption test was performed to assess the impact of the carboxybetaine species on the protein adsorption behaviour of the PTFE surface, in which the fluoropolymer surface of the cannula was treated with a bovine serum albumin (BSA) protein solution. BSA adsorption was assessed by fluorescence after 24- and 72-hours treatment.

The functionalised cannula demonstrated minimal fluorescence after both 24 and 72 hours of treatment, which suggested minimal protein adsorption had occurred on the functionalised surface. This was in stark contrast to an unmodified PTFE cannula control, which displayed substantial fluorescence after both time periods.

These results highlight the excellent protein-repellent properties provided by the carboxybetaine functionalised PTFE surface. The mechanical properties of the PTFE cannula were not negatively impacted, and the PTFE retained its lubricious non-stick surface.

### EXAMPLE 2

A second embodiment of a medical device of the first aspect of the invention is provided by a cannula containing a polymeric tubular body having a PTFE outer surface. The PTFE outer surface is functionalised with a sulfobetaine polymer derived from 3-[[2-(Methacryloyloxy)ethyl]dimethylammonio]propane-1-sulfonate monomer units, which is bonded to the PTFE surface via a linker derived from glycidyl methacrylate.

The cannula is part of a patch pump of the second aspect of the invention for the subcutaneous delivery of insulin. A cross-sectional view of the patch pump is displayed in FIG. 3. With reference to FIG. 3, the patch pump comprises a body (101), which is attachable to the skin of a user via an adhesive part of the body (101). The patch pump comprises a cannula (105) which extends from the body (101).

The body (101) comprises a fluid part (107), which is part of the body and provides a fluid path through the patch pump, allowing for fluid communication between the body (101) and the cannula (105). The fluid part (107) also contains a cartridge of insulin (not shown) for subcutaneous delivery. The arrangement also allows for fluid communication between the inside of the insulin cartridge and the cannula (105).

The body (101) further comprises an inbuilt pump (not shown).

In use, the body (101) of the patch pump is attached to the skin of the user via the adhesive part of the body (101). The free end of the cannula (105), which projects from the body (101), is inserted into the body of the user. Insulin is delivered subcutaneously from the patch pump via the cannula (105), with assistance from the inbuilt pump.

### Cannula Preparation Method

The functionalised cannula was prepared as follows.

Surface Activation: surface activation was performed as described for Example 1 above.

Linker functionalisation: the cannula was thereafter submerged in a solution of glycidyl methacrylate (2 wt % in methyl tert-butyl ether) at room temperature for 1 hour. The cannula was then removed, rinsed with deionised water at ambient temperature, and sonicated for 10 minutes in fresh deionised water. The cannula was then air dried.

Sulfobetaine functionalisation: a solution containing 1 wt % of 3-[[2-(Methacryloyloxy)ethyl]dimethylammonio]propane-1-sulfonate and 2 wt % of AIBN polymerisation initiator was prepared in a methanol solvent. The prepared solution was held at 50° C. for 30-60 minutes prior to use to initiate polymerisation. The cannula was submerged in the prepared solution at 50° C. for 1 hour, prior to rinsing with water, sonicating and air drying, as performed previously. Submerging the cannula in the solution allowed for covalent linkage of sulfobetaine polymer chains to the linker derived from glycidyl methacrylate which was present on the fluoropolymer surface.

### Results

The final functionalised cannula contained a thin layer of sulfobetaine polymer adsorbed to the fluoropolymer surface via a linker.

A protein adsorption test was performed as in Example 1, and results achieved were similar.

The mechanical properties of the PTFE cannula were not negatively impacted, and the PTFE retained its lubricious non-stick surface.

The above embodiments are described by way of example only. Many variations are possible without departing from the scope of the invention as defined in the appended claims.

1. A medical device comprising a fluoropolymer surface comprising at least one species independently chosen from: a carboxybetaine, a sulfobetaine, and combinations and/or polymers thereof.

2. A medical device as claimed in claim 1, wherein the at least one species is present as a coating on the fluoropolymer surface.

3. A medical device as claimed in claim 1 or 2, wherein the fluoropolymer is independently chosen from: polytetrafluoroethylene, polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof, and wherein the fluoropolymer preferably comprises polytetrafluoroethylene.

4. A medical device as claimed in any preceding claim, wherein the at least one species comprises a carboxybetaine and/or a polymer thereof.

5. A medical device as claimed in any preceding claim, wherein the at least one species comprises at least one poly(carboxybetaine) and/or at least one poly(sulfobetaine) polymer.

6. A medical device as claimed in claim 5, wherein the at least one poly(carboxybetaine) and/or poly(sulfobetaine) polymer comprises at least one acrylate and/or alkacrylate polymer.

7. A medical device as claimed in claim 5 or 6, wherein the at least one species comprises at least on poly(carboxybetaine) comprising at least one repeat unit derived from an unsaturated monomer of Formula (I): 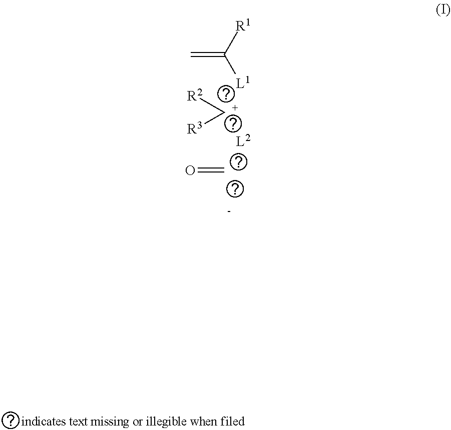
wherein: R¹ is:
H; a straight or branched C1-C10 alkyl; C1-C12 aryl; or halogen;
R² and R³, which may be the same or different, are:
a straight or branched C1-C10 alkyl; or a C1-C12 aryl;
L¹ is:
a linker that covalently couples the ammonium group to the unsaturated polymerizable moiety;
L² is:
a linker that covalently couples the ammonium group to the carboxylate group.

8. A medical device as claimed in claim 7, wherein R¹, R² and R³ are the same, and preferably methyl.

9. A medical device as claimed in claim 7 or 8, wherein L¹ is: —C(═O)X—(CH₂)ₙ—; wherein X is: N or O; and n is: 1-5, preferably 1-3, and more preferably 1-2.

10. A medical device as claimed in any one of claims 7 to 9, wherein L² is: a straight-chained C1-C5 alkyl, preferably C1-C3 alkyl, and more preferably C1-C2 alkyl.

11. A medical device as claimed in any preceding claim, wherein the at least one species is adsorbed to the fluoropolymer surface, and wherein the at least one species is preferably physisorbed and/or chemisorbed to the fluoropolymer surface.

12. A medical device as claimed in any preceding claim, wherein the at least one species is covalently and/or ionically/electrostatically bonded to the fluoropolymer surface.

13. A medical device as claimed in any preceding claim, wherein the at least one species is bonded to the fluoropolymer surface via a linker.

14. A medical device as claimed in claim 13, wherein the linker is derived from a linking compound comprising a bi- or poly-functional molecule comprising at least two reactive functional groups, and wherein the linker is preferably derived from a linking compound comprising an electrophilic moiety and a polymerizable moiety, preferably a polymerizable unsaturated group.

15. A medical device as claimed in any preceding claim, wherein the at least one species is present at a total concentration of at least 0.5 wt. % of the medical device.

16. A medical device as claimed in claim 15, wherein the at least one species is present at a total concentration of between 0.5-15 wt. %, or between 0.5-5 wt. % of the medical device.

17. A medical device as claimed in any preceding claim, wherein the medical device comprises a tubular body comprising the fluoropolymer surface, and wherein the fluoropolymer surface preferably comprises an outer surface of the tubular body, and preferably comprises at least 70% of the outer surface area of the tubular body.

18. A medical device as claimed in any preceding claim, wherein the medical device is a cannula or a catheter.

19. A medical device as claimed in any claim 18, wherein the medical device is a cannula that is part of an infusion set or patch pump.

20. An infusion set or patch pump comprising a cannula comprising a fluoropolymer surface comprising at least one species independently chosen from: a carboxybetaine, a sulfobetaine, and combinations and/or polymers thereof.

21. A method of manufacturing a medical device according to any one of claims 1 to 19, the method comprising the steps of:
a. Providing a medical device comprising a fluoropolymer surface;
b. Activating the fluoropolymer surface; and
c. Functionalising the activated fluoropolymer surface with at least one species independently chosen from: a carboxybetaine, a sulfobetaine, and
combinations and/or polymers thereof.

22. A method as claimed in claim 21, wherein steps (b) and (c) are performed simultaneously.

23. A method as claimed in claim 21, wherein step (c) is performed subsequently to step (b).

24. A method as claimed in any one of claims 21 to 23, wherein step (b) comprises producing an activated fluoropolymer surface comprising at least one unsaturated reactive moiety.

25. A method as claimed in any one of claims 21 to 24, wherein step (b) comprises the step of plasma treating the fluoropolymer surface.

26. A method as claimed in any one of claims 21 to 25, wherein step (b) comprises the step of treating the fluoropolymer surface with at least one reducing agent.

27. A method as claimed in claim 26, wherein the at least one reducing agent is dissolved in a carrier solvent comprising a glycol ether, preferably an aprotic glycol ether.

28. A method as claimed in claim 26 or 27, wherein step (b) comprises treating the fluoropolymer surface with the at least one reducing agent at a temperature of between 45-65° C., or between 50-65° C.

29. Use of at least one species that is independently chosen from: a carboxybetaine, a sulfobetaine, and combinations and/or polymers thereof, as a protein-repellent in and/or on a medical device.
